(19) United States
(12) Patent Application Publication
(10) Pub. No.: US 2023/0226043 A1
(43) Pub. Date: Jul. 20, 2023

(54) USE OF MASITINIB FOR THE TREATMENT OF CORONAVIRUS DISEASE 2019 (COVID-19)

(71) Applicants: AB SCIENCE, Paris (FR); THE UNIVERSITY OF CHICAGO, Chicago, IL (US)

(72) Inventors: Alain MOUSSY, Paris (FR); Savas TAY, Chicago, IL (US); Nir DRAYMAN, Evanston, IL (US); Glenn RANDALL, Chicago, IL (US); Siquan CHEN, Burlingame, CA (US)

(73) Assignees: AB SCIENCE, Paris (FR); THE UNIVERSITY OF CHICAGO, Chicago, IL (US)

(21) Appl. No.: 17/917,438

(22) PCT Filed: Apr. 9, 2021
(86) PCT No.: PCT/EP2021/059355

§ 371 Date: Oct. 6, 2022

Related U.S. Application Data
(60) Provisional application No. 63/062,775, filed on Aug. 7, 2020.
(60) Provisional application No. 63/127,436, filed on Dec. 18, 2020.

(30) Foreign Application Priority Data
Apr. 10, 2020 (EP) 20315155.0

(51) Int. Cl. A61K 31/496; A61K 31/352; A61K 45/06; A61P 31/14

(52) U.S. Cl. CPC A61K 31/496; A61K 31/352; A61K 45/06; A61P 31/14

(57) ABSTRACT

A method for the treatment of a coronavirus infection, such as a SARS-CoV-2 infection causing coronavirus disease 2019 (COVID-19), in a subject in need thereof, which includes the administration of masitinib, or a pharmaceutically acceptable salt or solvate thereof. In addition, at least one further pharmaceutically active agent may also be administered.

### FIELD OF INVENTION

The present invention relates to the treatment of a coronavirus infection in a subject in need thereof. In particular, the present invention relates to the treatment of a SARS-CoV-2 infection in a subject in need thereof, that is to say to the treatment of COVID-19, COVID-19 associated pneumonia and/or COVID-19 associated acute respiratory distress syndrome (ARDS) in a subject in need thereof.

### BACKGROUND OF INVENTION

Coronaviruses (CoVs) are positive-sense single-stranded ribonucleic acid (RNA) viruses (+ssRNA viruses) of the Coronaviridae family, characterized by an unusually large RNA genome, a unique replication strategy and a distinctive morphology as seen by electron microscopy, i.e., a crownlike appearance resulting from club-shaped spikes projecting from the surface of their envelope (Fehr & Perlman, Methods Mol Biol. 2015; 1282:1-23). Coronaviruses, which are nidoviruses, (i.e., they belong to the order Nidovirales) infect mammals and birds and cause a wide range of respiratory, gastrointestinal, neurologic, and systemic diseases.

Human coronaviruses were first identified in the mid-1960 and were initially thought to cause only mild respiratory infections in most cases, such as the common cold. Four endemic human CoVs (HCoV-229E, HCoV-NL63, HCoV-OC43, HCoV-HKU1) are thus estimated to account for 10% to 30% of upper respiratory tract infections in human adults (Paules et al., JAMA. 2020 Feb. 25; 323(8):707-708). However, in recent years, two highly pathogenic coronaviruses causing severe respiratory diseases emerged from animal reservoirs: severe acute respiratory syndrome coronavirus (SARS-CoV) first identified in 2003 and Middle East respiratory syndrome coronavirus (MERS-CoV) first identified in 2012. 8096 cases of severe acute respiratory syndrome (SARS) were reported world-wide, including 774 deaths and 1728 cases of Middle East respiratory syndrome (MERS) were reported world-wide, including 624 deaths (de Wit et al., Nat Rev Microbiol. 2016; 14(8):523-534).

In December 2019, the Wuhan Municipal Health Committee (Wuhan, China) identified a new infectious respiratory disease of unknown cause (Huang et al., Lancet. 2020; 395(10223):497-506; Wang et al., Lancet. 2020; 395(10223):470-473; Zhu et al., N Engl J Med. 2020; 382(8):727-733). Coronavirus RNA was quickly identified in some of the patients and in January 2020, researchers from the Shanghai Public Health Clinical Center & School of Public Health and their collaborators released a full genomic sequence of the newly identified human coronavirus SARS-CoV-2 (previously known as 2019-nCoV). The genomic sequence of SARS-COV-2 has 89% nucleotide identity with the genomic sequence of bat coronavirus SARS-like-CoVZXC21 and 82% nucleotide identity with the genomic sequence of human SARS-CoV (Chan et al., Lancet. 2020; 395(10223):514-523). As previously shown for SARS-CoV, SARS-CoV2 appears to utilize ACE2 (angiotensin converting enzyme 2) as receptor for viral cell entry (Hoffmann et al., Cell. 2020 Apr. 16; 181(2):271-280).

SARS-CoV2 infection is thought to be asymptomatic or causing little or no clinical manifestations in 30 to 60% of infected subjects. In infected subjects with symptoms, the disease caused by SARS-COV-2 is now termed “coronavirus disease 2019” (COVID-19). COVID-19 is a respiratory illness generally first presenting with symptoms including headache, muscle pain, and/or fatigue/tiredness followed by fever and respiratory symptoms (such as a dry cough, shortness of breath, and/or chest tightness). While the symptoms remain mild in the majority of subjects, in others they may progress to pneumonia (referred herein as COVID-19 associated pneumonia or COVID-19 pneumonia) and/or to multi-organ failure. Complications of COVID-19 include acute respiratory distress syndrome (ARDS) (referred herein as COVID-19 associated ARDS or COVID-19 ARDS), RNAaemia, acute cardiac injury and secondary infections (Huang et al., Lancet. 2020; 395(10223):497-506). It is estimated that about 5% of subjects suffering from COVID-19 require hospitalization, among which about 25% require admission to intensive care unit (ICU). COVID-19 causes substantial morbidity and mortality and may place unprecedented strain on many health systems.

Global efforts to evaluate novel antivirals and therapeutic strategies to treat COVID-19 have thus intensified. Notably, a number of clinical trials have been registered to assess the efficacy of drugs such as, for example, remdesivir (a nucleotide analog antiviral under development), lopinavir/ritonavir (an antiretroviral therapy notably used for the treatment of human immunodeficiency virus 1 (HIV-1)), and chloroquine or hydroxychloroquine (both notably used for the prevention and treatment of malaria, and also for the treatment of rheumatoid arthritis and lupus erythematosus). However, there remains a lack of therapeutic agents with a proven efficacy for preventing and/or treating COVID-19, COVID-19 associated pneumonia or COVID-19 associated acute respiratory distress syndrome (ARDS).

Therefore, there is still a need for effective treatments for infections with nidoviruses, including coronaviruses, as well as for infections with picornaviruses, which are +ssRNA viruses belonging to the same class than nidoviruses (i.e., the Pisoniviricetes class). In particular, there is still a need for effective treatments for coronavirus infections, notably for beta (3) coronavirus infections. Currently, there is an urgent need for effective and safe treatments for SARS-CoV-2 infection causing COVID-19, in particular prophylactic treatments and/or therapeutic treatments for COVID-19 associated pneumonia and COVID-19 associated acute respiratory distress syndrome (ARDS).

The present invention relates to a 2-aminoarylthiazole derivative, preferably masitinib, or a pharmaceutically acceptable salt or solvate thereof, optionally in combination with isoquercetin, for use in the treatment of a nidovirus or a picornavirus infection, in particular for use in the treatment of a coronavirus infection, such as a SARS-CoV-2 infection causing COVID-19, in a subject in need thereof.

### SUMMARY

The present invention relates to a 2-aminoarylthiazole derivative, or a pharmaceutically acceptable salt or solvate thereof, for use in the treatment of a coronavirus infection in a subject in need thereof.

In one embodiment, the coronavirus infection is a severe acute respiratory syndrome coronavirus 2 (SARS-CoV-2) infection causing coronavirus disease 2019 (COVID-19).

In one embodiment, the 2-aminoarylthiazole derivative, or a pharmaceutically acceptable salt or solvate thereof, is for administration in combination with isoquercetin, preferably for administration in combination with a dose of isoquercetin ranging from about 0.4 g/day to about 2 g/day, more preferably for administration in combination with a dose of isoquercetin of about 1 g/day.

In one embodiment, the 2-aminoarylthiazole derivative has the formula (II):

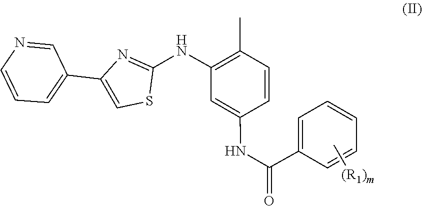

wherein:
R₁ is selected independently from hydrogen, halogen, (C₁-C₁₀) alkyl, (C₃-C₁₀) cycloalkyl group, trifluoromethyl, alkoxy, amino, alkylamino, dialkylamino, a solubilizing group, and (C₁-C₁₀) alkyl substituted by a solubilizing group; and
m is 0-5.

In one embodiment, the 2-aminoarylthiazole derivative, or a pharmaceutically acceptable salt or solvate thereof, is masitinib or a pharmaceutically acceptable salt or solvate thereof. In one embodiment, the pharmaceutically acceptable salt of masitinib is masitinib mesilate.

In one embodiment, the 2-aminoarylthiazole derivative, or a pharmaceutically acceptable salt or solvate thereof, is for oral administration. In one embodiment, the 2-aminoarylthiazole derivative, or a pharmaceutically acceptable salt or solvate thereof, is for administration at a dose ranging from about 1 mg/kg/day to about 12 mg/kg/day (mg per kilo body weight per day), preferably at a dose ranging from about 3 mg/kg/day to about 6 mg/kg/day. In one embodiment, the 2-aminoarylthiazole derivative, or a pharmaceutically acceptable salt or solvate thereof, is for administration at an initial dose of about 3 mg/kg/day during at least one week, and at a dose of about 4.5 mg/kg/day thereafter, with each dose escalation being subjected to toxicity controls.

In one embodiment, the subject presents at least one risk factor that may lead to an increased risk of developing COVID-19.

In one embodiment, the subject is suffering from mild-to-moderate COVID-19, preferably from moderate COVID-19. In one embodiment, the subject is suffering from severe COVID-19. In one embodiment, the subject is suffering from critical COVID-19.

In one embodiment, the subject is suffering from COVID-19 and has a score on the World Health Organization (WHO) 10-point progression scale of COVID-19 (as described in Table 1 herein) ranging from 2 to 9. In one embodiment, the subject is suffering from COVID-19 and has a score on the WHO 10-point progression scale of COVID-19 (as described in Table 1 herein) of 2 or 3. In one embodiment, the subject is suffering from COVID-19 and has a score on the WHO 10-point progression scale of COVID-19 (as described in Table 1 herein) ranging from 4 to 6, preferably of 4 or 5. In one embodiment, the subject is suffering from COVID-19 and has a score on the modified WHO 7-point progression scale of COVID-19 (as described in Table 2 herein) ranging from 2 to 6, preferably ranging from 2 to 5, more preferably of 4 or 5.

In one embodiment, the 2-aminoarylthiazole derivative, or a pharmaceutically acceptable salt or solvate thereof, is for administration with at least one further pharmaceutically active agent. In one embodiment, the at least one further pharmaceutically active agent is selected from the group consisting of antiviral agents, anti-interleukin 6 (anti-IL6) agents, protease inhibitors, Janus-associated kinase (JAK) inhibitors, BXT-25, brilacidin, dehydroandrographolide succinate, APN01, fingolimod, methylprednisolone, thalidomide, bevacizumab, sildenafil citrate, interferon, carrimycin, and any mixes thereof.

### Definitions

In the present invention, the following terms have the following meanings:

“About” preceding a figure encompasses plus or minus 10%, or less, of the value of said figure. It is to be understood that the value to which the term “about” refers is itself also specifically, and preferably, disclosed.

“Baseline” as used herein refers to the time preceding the start of the treatment with the 2-aminoarylthiazole derivative, preferably masitinib, or a pharmaceutically acceptable salt or solvate thereof, as described herein. For example, for a given subject, interleukin 6 (IL6) plasma levels at baseline are the interleukin 6 (IL6) plasma levels prior to the administration to the subject of a 2-aminoarylthiazole derivative, preferably masitinib, or a pharmaceutically acceptable salt or solvate thereof, as described herein.

“Best supportive care” refers to the supportive care routinely provided to a subject hospitalized and suffering from a respiratory illness, in particular a lower tract respiratory illness, such as pneumonia or ARDS. Best supportive care may include for example at least one of the following: supplemental oxygen (02) also referred to as oxygen therapy (for example by mask or nasal prongs), non-invasive ventilation (NIV), invasive mechanical ventilation, extracorporeal membrane oxygenation (ECMO), vasopressor therapy (such as for example phenylephrine, norepinephrine, epinephrine, vasopressin, and/or dopamine), fluid therapy, antimicrobial therapy, renal support, sedation.

“Consisting essentially of” as used herein with reference to a composition, pharmaceutical composition or medicament, is intended to mean that the 2-aminoarylthiazole derivative as described herein, preferably masitinib, or a pharmaceutically acceptable salt or solvate thereof, is the only active agent (also referred to as active ingredient or as active compound), i.e., the only agent exhibiting a biological or pharmacological activity, within said composition, pharmaceutical composition or medicament.

“High flow” or “high flow oxygen” as used herein refers to high flow oxygen therapy (HFOT) which is a form of respiratory support.

“Laboratory confirmed SARS-CoV-2 infection” as used herein refers to a SARS-CoV-2 infection confirmed by a laboratory test such as a rRT-PCR (real-time reverse transcription polymerase chain reaction) test allowing to detect the presence of SARS-CoV-2 in a sample from a subject (such as a sample from a nasal swab, a sample from an oropharyngeal swab, a sputum sample, a lower respiratory tract aspirate, a bronchoalveolar lavage, a nasopharyngeal wash/aspirate or a nasal aspirate) or an antibody test (such as an enzyme-linked immunosorbent assay (ELISA)) allowing to detect the presence of antibodies against SARS-CoV-2 in a sample from a subject (such as a blood sample).

“Pharmaceutically acceptable excipient” or “pharmaceutically acceptable carrier” refers to an excipient or carrier that does not produce an adverse, allergic or other untoward reaction when administered to a mammal, preferably a human. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).

“Subject” refers to a mammal, preferably a human. Mammals include, but are not limited to, the order Rodentia, including mice; the order Lagomorpha, including rabbits; the order Carnivora, including felines (cats) and canines (dogs); the order Artiodactyla, including bovines (cows) and swines (pigs); the order Perissodactyla, including equines (horses); the order Primates, including monkeys, apes and humans. In one embodiment, the mammal is selected from Rodentia, Lagomorpha, Carnivora, Artiodactyla, Perissodactyla, and Primates. In one embodiment, the mammal is selected from mice, rabbits, cats, dogs, cows, pigs, horses, monkeys, apes and humans. In one embodiment, the subject is a primate, preferably a human. According to one embodiment, the subject is a mammal, preferably a human, having come in contact with, suspected to have come in contact with, or expected to come into contact with a nidovirus or a picornavirus, in particular with a coronavirus such as SARS-CoV-2. According to one embodiment, the subject is a mammal, preferably a human, suffering from a nidovirus infection or a picornavirus infection, preferably from a coronavirus infection, in particular from a SARS-CoV-2 infection causing COVID-19. In one embodiment, the subject may be a “patient”, i.e., a mammal, in particular a warm-blooded mammal, preferably a human, who/which is awaiting the receipt of, or is receiving medical care or was/is/will be the object of a medical procedure, or is monitored for the development of a nidovirus infection or a picornavirus infection, preferably a coronavirus infection, in particular a SARS-CoV-2 infection causing COVID-19.

“Therapeutically effective amount” or “therapeutically effective dose” refers to the amount or dose or concentration of a 2-aminoarylthiazole derivative as described herein, preferably masitinib, or a pharmaceutically acceptable salt or solvate thereof, optionally in combination with isoquercetin or quercetin, sufficient to induce a meaningful benefit in a subject, cell, or tissue to be treated. A meaningful benefit includes, for example, detectably treating, relieving, or lessening one or more symptoms of a disease caused by a nidovirus or picornavirus (e.g., inflammation, fluid accumulation), in particular by a coronavirus; inhibiting, arresting development, preventing, or halting further development of the viral infection or disease caused by a nidovirus or picornavirus, in particular by a coronavirus; reducing the incidence of a disease caused by a nidovirus or picornavirus, in particular by a coronavirus; preventing a disease caused by a nidovirus or picornavirus, in particular by a coronavirus, from occurring in a subject, cell, or tissue at risk thereof but yet to be diagnosed; and/or detectably inhibiting one or more active sites of viral proteins in a subject, cell, or tissue. The meaningful benefit observed in the subject, cell, or tissue to be treated may be to any suitable degree (10, 20, 30, 40, 50, 60, 70, 80, 90% or more). In one embodiment, the therapeutically effective dose is the amount or dose or concentration of a 2-aminoarylthiazole derivative as described herein, preferably masitinib, or a pharmaceutically acceptable salt or solvate thereof, optionally in combination with isoquercetin or quercetin, that is aimed at, without causing significant negative or adverse side effects to the subject in need of treatment, preventing, reducing, alleviating or slowing down (lessening) one or more of the symptoms or manifestations of a nidovirus infection or a picornavirus infection, preferably a coronavirus infection, in particular of a SARS-CoV-2 infection causing COVID-19, in said subject.

“Treating” or “Treatment” refers to a therapeutic treatment, to a prophylactic (or preventative) treatment, or to both a therapeutic treatment and a prophylactic (or preventative) treatment, wherein the object is to prevent, reduce, alleviate, and/or slow down (lessen) one or more of the symptoms or manifestations of a nidovirus infection or a picornavirus infection, preferably a coronavirus infection, in particular of a SARS-CoV-2 infection causing COVID-19, in a subject in need thereof. Symptoms of a coronavirus infection, in particular of a SARS-CoV-2 infection causing COVID-19, include, without being limited to, a fever and respiratory symptoms such as dry cough and/or breathing difficulties that may require respiratory support (for example supplemental oxygen, non-invasive ventilation, invasive mechanical ventilation, extracorporeal membrane oxygenation (ECMO)). Manifestations of a coronavirus infection, in particular of a SARS-CoV-2 infection, include, without being limited to, the viral load (also known as viral burden or viral titer) detected in a sample from the subject. In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventive) treatment and a therapeutic treatment. In one embodiment, the object of the treatment according to the present application is to bring about at least one of the following:
a reduction in the viral load detected in a sample from the subject;
a decrease in the requirement for respiratory support, for example a decrease in the use of ECMO, invasive mechanical ventilation, non-invasive ventilation, or supplemental oxygen including high flow oxygen therapy; and/or a decrease in the requirement for vasopressor therapy;
a discharge from the intensive care unit;
a discharge from hospital.

### DETAILED DESCRIPTION

The present invention relates to a 2-aminoarylthiazole derivative as described herein, in particular masitinib, or a pharmaceutically acceptable salt or solvate thereof, for use in the treatment of a nidovirus infection in a subject in need thereof. Examples of nidoviruses (i.e., viruses belonging to the order Nidovirales) include coronaviruses, toroviruses, arteriviruses, and okaviruses. Diseases caused by a nidovirus include, without being limited to, coronavirus disease 2019 (COVID-19), severe acute respiratory syndrome (SARS), Middle East respiratory syndrome (MERS), a respiratory disease (e.g., pneumonia, bronchitis, pleural effusion), an inflammatory disease (e.g., inflammation, COVID-19-induced inflammation, pediatric multisystem inflammatory syndrome (PMIS)), porcine reproductive and respiratory syndrome, equine viral arteritis, and gastroenteritis).

In one embodiment, the nidovirus is a coronavirus, an arterivirus, or a torovirus. In one embodiment, the nidovirus is a coronavirus.

According to one embodiment, the present invention thus relates to a 2-aminoarylthiazole derivative as described herein, in particular masitinib, or a pharmaceutically acceptable salt or solvate thereof, for use in the treatment of a coronavirus infection in a subject in need thereof.

In one embodiment, the coronavirus is an alpha (a) coronavirus or a beta (P) coronavirus, preferably a beta coronavirus, including βA coronaviruses, βB coronaviruses, βC coronaviruses, and βD coronaviruses. Thus, in one embodiment, the coronavirus is a beta (P) coronavirus. In one embodiment, the beta (P) coronavirus is a βA, βB, βC, or βD coronavirus.

Examples of alpha coronaviruses include, without being limited to, human coronavirus 229E (HCoV-229E) and human coronavirus NL63 (HCoV-NL63) also sometimes known as HCoV-NH or New Haven human coronavirus. Examples of beta coronaviruses include, without being limited to, human coronavirus OC43 (HCoV-OC43), human coronavirus HKU1 (HCoV-HKU1), Middle East respiratory syndrome-related coronavirus (MERS-CoV) previously known as novel coronavirus 2012 or HCoV-EMC, severe acute respiratory syndrome coronavirus (SARS-CoV) also known as SARS-CoV-1 or SARS-classic, and severe acute respiratory syndrome coronavirus (SARS-CoV-2) also known as 2019-nCoV or novel coronavirus 2019. In one embodiment, the beta (P) coronavirus is HCoV-OC43, MERS-CoV, SARS-CoV (also known as SARS-CoV-1), or SARS-CoV-2. In one embodiment, the beta (P) coronavirus is HCoV-sOC43 or SARS-CoV-2.

In one embodiment, the coronavirus is selected from the group comprising or consisting of HCoV-229E, HCoV-NL63, HCoV-OC43, HCoV-HKU1, MERS-CoV, SARS-CoV-1 and SARS-CoV-2.

In one embodiment, the coronavirus is selected from the group comprising or consisting of MERS-CoV, SARS-CoV-1 and SARS-CoV-2. Thus, in one embodiment, the present invention relates to a 2-aminoarylthiazole derivative as described herein, or a pharmaceutically acceptable salt or solvate thereof, for use in the treatment of a MERS-CoV coronavirus infection causing MERS, a SARS-CoV-1 infection causing SARS or a SARS-CoV-2 infection causing COVID-19 in a subject in need thereof.

In one embodiment, the coronavirus is a MERS coronavirus. In one embodiment, the coronavirus is MERS-CoV causing Middle East respiratory syndrome (MERS).

In one embodiment, the coronavirus is a SARS coronavirus.

In one embodiment, the coronavirus is SARS-CoV-1 or SARS-CoV-2. Thus, in one embodiment, the present invention relates to a 2-aminoarylthiazole derivative as described herein, or a pharmaceutically acceptable salt or solvate thereof, for use in the treatment of a SARS-CoV-1 infection causing SARS or a SARS-CoV-2 infection causing COVID-19 in a subject in need thereof.

In one embodiment, the coronavirus is SARS-CoV (also referred to as SARS-CoV-1) causing severe acute respiratory syndrome (SARS).

According to one embodiment, the coronavirus is SARS-CoV-2 causing COVID-19. Thus, according to one embodiment, the present invention relates to a 2-aminoarylthiazole derivative as described herein, in particular masitinib, or a pharmaceutically acceptable salt or solvate thereof, for use in the treatment of a SARS-CoV-2 infection causing COVID-19 in a subject in need thereof. In one embodiment, the present invention relates to a 2-aminoarylthiazole derivative as described herein, in particular masitinib, or a pharmaceutically acceptable salt or solvate thereof, for use in the treatment of COVID-19 in a subject in need thereof.

The present invention also relates to a 2-aminoarylthiazole derivative as described herein, in particular masitinib, or a pharmaceutically acceptable salt or solvate thereof, for use in the treatment of a picornavirus infection in a subject in need thereof. Examples of picornaviruses include polioviruses, rhinoviruses, enteroviruses, and coxsackieviruses. Diseases caused by a picornavirus include, without being limited to, acute flaccid myelitis (AFM), respiratory diseases, and gastrointestinal diseases.

In one embodiment, the picornavirus is a poliovirus, a rhinovirus, an enterovirus, or a coxsackievirus. In one embodiment, the picornavirus is a rhinovirus or a coxsackievirus.

The present invention thus relates to a 2-aminoarylthiazole derivative as described herein, in particular masitinib, or a pharmaceutically acceptable salt or solvate thereof, for use in the treatment of a nidovirus infection or a picornavirus infection in a subject in need thereof. In other words, the present invention thus relates to a 2-aminoarylthiazole derivative as described herein, in particular masitinib, or a pharmaceutically acceptable salt or solvate thereof, for use in the treatment of an infection with a virus of the Pisoniviricetes class, wherein said virus of the Pisoniviricetes class is a nidovirus or a picornavirus.

In one embodiment, COVID-19 severity is assessed according to the diagnostic and treatment guideline for SARS-CoV-2 issued by the Chinese National Health Committee (Chen et al., Detectable serum SARS-CoV-2 viral load (RNAaemia) is closely associated with drastically elevated interleukin 6 (IL-6) level in critically ill COVID-19 patients. medRxiv 2020.02.29.20029520; Liu et al., The potential role of IL-6 in monitoring severe case of coronavirus disease 2019. medRxiv 2020.03.01.20029769; Zhang et al., Allergy. 2020 July; 75(7):1730-1741).

In one embodiment, COVID-19 severity is assessed according to the Belgium National Public Health Institute (Sciensano) (Interim clinical guidance for patients suspected of/confirmed with COVID-19 in Belgium (19 Mar. 2020; Version 4), retrieved at https://epidemio.wiv-isp.be/ID/Documents/Covid19/COVID-19_InterimGuidelines_Treatment_ENG.pdf).

In one embodiment, COVID-19 severity is assessed according to the World Health Organization (WHO) criteria of severity. The WHO criteria of severity of COVID-19 are as follows:
mild: cases showing mild clinical symptoms with no sign of pneumonia on imaging.
moderate: cases showing fever and respiratory symptoms with radiological findings of pneumonia and requiring oxygen (O₂): 3 L/min<oxygen<5 L/min;
severe: cases meeting any of the following criteria:
   respiratory distress (respiratory rate (RR)≥30 breaths/min);
   oxygen saturation (SpO₂)≤93% at rest in ambient air; or SpO₂≤97% with O₂>5 L/min;
   ratio of artery partial pressure of oxygen/inspired oxygen fraction (PaO₂/FiO₂)≤300 mmHg (1 mmHg=0.133 kPa), PaO₂/FiO₂ in high-altitude areas (at an altitude of over 1,000 meters above the sea level) shall be corrected by the following formula: PaO₂/FiO₂ [multiplied by] [Atmospheric pressure (mmHg)/760]; and/or
   chest imaging that showed obvious lesion progression within 24-48 hours>50%;
critical: cases meeting any of the following criteria:
   respiratory failure and requiring mechanical ventilation;
   shock; and/or
   other organ failure that requires ICU care.

In one embodiment, COVID-19 severity and/or progression is assessed with the WHO 10-point progression scale as indicated in Table 1 below (WHO Working Group on the Clinical Characterisation and Management of COVID-19 infection. A minimal common outcome measure set for COVID-19 clinical research. Lancet Infect Dis. 2020 August; 20(8):e192-e197. doi: 10.1016/S1473-3099(20)30483-7).

TABLE 1
WHO 10-point progression scale of COVID-19

| WHO 10-point | | |
|---|---|---|
| Progression scale | Descriptor | Score |
| Uninfected | Uninfected; no viral | 0 |
| | RNA detected | |
| Ambulatory: mild disease | Asymptomatic; viral | 1 |
| | RNA detected | |
| Ambulatory: mild disease | Symptomatic; independent | 2 |
| Ambulatory: mild disease | Symptomatic; assistance needed | 3 |
| Hospitalized: moderate disease | Hospitalized; no oxygen therapy | 4 |
| Hospitalized: moderate disease | Hospitalized; oxygen by mask or | 5 |
| | nasal prongs | |
| Hospitalized: severe disease | Hospitalized; oxygen by non- | 6 |
| | invasive ventilation (NIV) | |
| | or high flow | |
| Hospitalized: severe disease | Intubation and mechanical | 7 |
| | ventilation, PaO₂/FIO₂ ≥150 | |
| | mmHg or oxygen saturation to | |
| | fraction of inspired oxygen ratio | |
| | (SpO₂/FIO₂) ≥200 mmHg | |
| Hospitalized: severe disease | Mechanical ventilation, | 8 |
| | PaO₂/FIO₂ <150 mmHg | |
| | or SpO₂/FIO₂ <200 | |
| | mmHg, or vasopressors | |
| | (norepinephrine | |
| | >0.3 μg/kg/min) | |
| Hospitalized: severe disease | Mechanical ventilation, | 9 |
| | PaO₂/FIO₂ <150 mmHg | |
| | and vasopressors | |
| | (norepinephrine >0.3 μg/ | |
| | kg/min), or dialysis or ECMO | |
| Death | Dead | 10 |

In one embodiment, the subject to be treated according to the present invention is suffering from COVID-19 and has a score on the WHO 10-point progression scale of COVID-19 (as described in Table 1) ranging from 2 to 9. In one embodiment, the subject to be treated according to the present invention is suffering from COVID-19 and has a score on the WHO 10-point progression scale of COVID-19 (as described in Table 1) ranging from 2 to 5. In one embodiment, the subject to be treated according to the present invention is suffering from COVID-19 and has a score on the WHO 10-point progression scale of COVID-19 (as described in Table 1) of 2, 3, 4, or 5. In one embodiment, the subject to be treated according to the present invention is suffering from COVID-19 and has a score on the WHO 10-point progression scale of COVID-19 (as described in Table 1) of 2 or 3. In one embodiment, the subject to be treated according to the present invention is suffering from COVID-19 and has a score on the WHO 10-point progression scale of COVID-19 (as described in Table 1) ranging from 4 to 9, preferably ranging from 4 to 6, more preferably of 4 or 5. In one embodiment, the subject to be treated according to the present invention is suffering from COVID-19 and has a score on the WHO 10-point progression scale of COVID-19 (as described in Table 1) of 4, 5 or 6.

In one embodiment, the subject to be treated according to the present invention is suffering from COVID-19 and is hospitalized, but does not require ICU at admission, and:
has a score on the WHO 10-point progression scale of COVID-19 (as described in Table 1) of 5; and
requires more than 3 L/min of oxygen but does not require non-invasive ventilation (NIV) or high flow.

In one embodiment, COVID-19 severity and/or progression is assessed with the modified WHO 7-point progression scale as indicated in Table 2 below.

TABLE 2
modified WHO 7-point progression scale of COVID-19

| Descriptor | Score |
|---|---|
| Not hospitalized, no limitations on activities | 1 |
| Not hospitalized, limitation on activities | 2 |
| Hospitalized, not requiring supplemental oxygen | 3 |
| Hospitalized, requiring supplemental oxygen | 4 |
| Hospitalized, on non-invasive ventilation (NIV) | 5 |
| or high flow oxygen devices | |
| Hospitalized, on invasive mechanical ventilation | 6 |
| or extracorporeal membrane oxygenation (ECMO) | |
| Death | 7 |

In one embodiment, the subject to be treated according to the present invention is suffering from COVID-19 and has a score on the modified WHO 7-point progression scale of COVID-19 (as described in Table 2) ranging from 2 to 6, preferably ranging from 2 to 5. In one embodiment, the subject to be treated according to the present invention is suffering from COVID-19 and has a score on the modified WHO 7-point progression scale of COVID-19 (as described in Table 2) ranging from 3 to 6, preferably ranging from 3 to 5. In one embodiment, the subject to be treated according to the present invention is suffering from COVID-19 and has a score on the modified WHO 7-point progression scale of COVID-19 (as described in Table 2) of 3, 4 or 5, preferably of 4 or 5.

In one embodiment, COVID-19 is mild-to-moderate COVID-19. Thus, in one embodiment, the present invention relates to a 2-aminoarylthiazole derivative as described herein, in particular masitinib, or a pharmaceutically acceptable salt or solvate thereof, for use in the prevention and/or treatment of mild-to-moderate COVID-19 in a subject in need thereof.

In one embodiment, mild-to-moderate COVID-19 is defined as a score on the WHO 10-point progression scale of COVID-19 (as described in Table 1) ranging from 1 to 5. In one embodiment, mild-to-moderate COVID-19 is defined as a score on the WHO 10-point progression scale of COVID-19 (as described in Table 1) of 1, 2, 3, 4, or 5.

In one embodiment, mild-to-moderate COVID-19 is defined as a laboratory confirmed SARS-CoV-2 infection associated with at least one of the following clinical symptoms: fever, respiratory symptoms (such as a cough, shortness of breath, and/or chest tightness), and imaging findings of pneumonia.

In one embodiment, the subject suffering from mild-to-moderate COVID-19 is not hospitalized. In one embodiment, the subject suffering from mild-to-moderate COVID-19 is hospitalized. In one embodiment, the subject suffering from mild-to-moderate COVID-19 is hospitalized but does not require admission to intensive care unit (ICU).

In one embodiment, the subject suffering from mild-to-moderate COVID-19 as described hereinabove requires oxygen therapy. In one embodiment, the subject suffering from mild-to-moderate COVID-19 as described hereinabove requires non-invasive ventilation (NIV).

In one embodiment, mild COVID-19 is defined as a laboratory confirmed SARS-CoV-2 infection with no oxygen (O₂) requirement or evidence of pneumonia.

In one embodiment, the subject suffering from mild COVID-19 is not hospitalized. In one embodiment, the subject suffering from mild COVID-19 is hospitalized. In one embodiment, the subject suffering from mild COVID-19 is hospitalized but does not require admission to ICU.

In one embodiment, mild COVID-19 is defined as a score on the WHO 10-point progression scale of COVID-19 (as described in Table 1) ranging from 1 to 3. In one embodiment, mild COVID-19 is defined as a score on the WHO 10-point progression scale of COVID-19 (as described in Table 1) of 1, 2, or 3. In one embodiment, mild COVID-19 is defined as a score on the WHO 10-point progression scale of COVID-19 (as described in Table 1) of 4 or 5.

In one embodiment, mild COVID-19 is defined as COVID-19 requiring hospitalization but no oxygen therapy. In one embodiment, mild COVID-19 is defined as COVID-19 requiring hospitalization and oxygen therapy by mask or nasal prongs.

In one embodiment, moderate COVID-19 is defined as a score on the WHO 10-point progression scale of COVID-19 (as described in Table 1) of 4 or 5. In one embodiment, moderate COVID-19 is defined as a score on the WHO 10-point progression scale of COVID-19 (as described in Table 1) of 5 with a requirement of more than 3 L/min of oxygen but without requirement of non-invasive ventilation (NIV) or high flow.

In one embodiment, moderate COVID-19 is defined as a laboratory confirmed SARS-CoV-2 infection associated with the following clinical symptoms: fever, respiratory symptoms (such as a dry cough, shortness of breath, and/or chest tightness), and imaging findings of pneumonia.

In one embodiment, the subject suffering from moderate COVID-19 is not hospitalized. In one embodiment, the subject suffering from moderate COVID-19 is hospitalized. In one embodiment, the subject suffering from moderate COVID-19 is hospitalized but does not require admission to ICU.

In one embodiment, the subject suffering from moderate COVID-19, defined as a score on the WHO 10-point progression scale of COVID-19 (as described in Table 1) of 5 with a requirement of more than 3 L/min of oxygen but without requirement of non-invasive ventilation (NIV) or high flow, is hospitalized but does not require admission to ICU.

In one embodiment, the subject suffering from moderate COVID-19 as described hereinabove requires oxygen therapy. In one embodiment, the subject suffering from moderate COVID-19 as described hereinabove requires NIV.

In one embodiment, COVID-19 is severe COVID-19. Thus, in one embodiment, the present invention relates to a 2-aminoarylthiazole derivative as described herein, in particular masitinib, or a pharmaceutically acceptable salt or solvate thereof, for use in the prevention and/or treatment of severe COVID-19 in a subject in need thereof.

In one embodiment, severe COVID-19 is defined as a laboratory confirmed SARS-CoV-2 infection associated with at least one of the following:
respiratory distress with respiratory frequency (or respiratory rate (RR))≥30/min;
pulse oximeter oxygen saturation ≤93% at rest; and/or
oxygenation index (ratio of artery partial pressure of oxygen/inspired oxygen fraction (PaO₂/FiO₂))≤300 mm Hg.

In one embodiment, severe COVID-19 is defined as a laboratory confirmed SARS-CoV-2 infection associated with at least one of the following:
shortness of breath, respiratory rate (RR)≥30 times/min;
oxygen saturation is less than 93% in resting state;
PaO₂/FiO₂≤300 mmHg; and/or
pulmonary lesion progressed more than 50% within 24 to 48 hours as evidenced by radiologic assessment.

In one embodiment, severe COVID-19 is defined as a laboratory confirmed SARS-CoV-2 infection associated with at least one of the following:
respiratory rate≥30/min (adults); ≥40/min (children <5);
blood oxygen saturation ≤93%:
PaO₂/FiO₂≤300 mmHg; and/or
lung infiltrates >50% of the lung field within 24-48 hours.

In one embodiment, severe COVID-19 is defined as a score on the WHO 10-point progression scale of COVID-19 (as described in Table 1) ranging from 6 to 9. In one embodiment, severe COVID-19 is defined as a score on the WHO 10-point progression scale of COVID-19 (as described in Table 1) of 6, 7, 8, or 9. In one embodiment, severe COVID-19 is defined as a score on the WHO 10-point progression scale of COVID-19 (as described in Table 1) of 6.

In one embodiment, severe COVID-19 is defined as COVID-19 requiring hospitalization and either NIV or high flow oxygen therapy.

In one embodiment, the subject suffering from severe COVID-19 is hospitalized.

In one embodiment, the subject suffering from severe COVID-19 is hospitalized but does not require admission to ICU. In one embodiment, the subject suffering from severe COVID-19 requires admission to ICU.

In one embodiment, the subject suffering from severe COVID-19 as described hereinabove requires oxygen therapy. In one embodiment, the subject suffering from severe COVID-19 as described hereinabove requires NIV.

In one embodiment, COVID-19 is critical COVID-19. Thus, in one embodiment, the present invention relates to a 2-aminoarylthiazole derivative as described herein, in particular masitinib, or a pharmaceutically acceptable salt or solvate thereof, for use in the prevention and/or treatment of critical COVID-19 in a subject in need thereof.

In one embodiment, critical COVID-19 is defined as a laboratory confirmed SARS-CoV-2 infection associated with at least one of the following, in addition to the criterion/criterion present in severe COVID-19:
respiratory failure requiring mechanical ventilation;
shock (septic shock); and/or
multiple organ failure (extra pulmonary organ failure) requiring admission to intensive care unit (ICU).

In one embodiment, critical COVID-19 is defined as a score on the WHO 10-point progression scale of COVID-19 (as described in Table 1) ranging from 7 to 9. In one embodiment, critical COVID-19 is defined as a score on the WHO 10-point progression scale of COVID-19 (as described in Table 1) of 7, 8 or 9.

In one embodiment, critical COVID-19 is defined as COVID-19 requiring hospitalization, intubation and mechanical ventilation, with PaO₂/FIO₂≥150 mmHg or SpO₂/FIO₂)≥200 mmHg.

In one embodiment, critical COVID-19 is defined as COVID-19 requiring hospitalization and one of the following:
mechanical ventilation (PaO₂/FIO₂<150 mmHg or SpO₂/FIO₂<200 mmHg); or
vasopressors (norepinephrine >0.3 μg/kg/min).

In one embodiment, critical COVID-19 is defined as COVID-19 requiring hospitalization and one the following:
mechanical ventilation (PaO₂/FIO₂<150 mmHg) and vasopressors (norepinephrine >0.3 μg/kg/min);
dialysis; or
ECMO.

In one embodiment, the subject suffering from critical COVID-19 is hospitalized. In one embodiment, the subject suffering from critical COVID-19 requires admission to ICU.

In one embodiment, the subject suffering from critical COVID-19 as described hereinabove requires oxygen therapy. In one embodiment, the subject suffering from critical COVID-19 as described hereinabove requires NIV. In one embodiment, the subject suffering from critical COVID-19 as described hereinabove requires invasive ventilation, such as intubation and mechanical ventilation. In one embodiment, the subject suffering from critical COVID-19 as described hereinabove requires vasopressor therapy (such as for example phenylephrine, norepinephrine, epinephrine, vasopressin, and/or dopamine).

In one embodiment, COVID-19 may lead to COVID-19 associated pneumonia (also referred to as COVID-19 pneumonia). Thus, in one embodiment, the present invention relates to a 2-aminoarylthiazole derivative as described herein, in particular masitinib, or a pharmaceutically acceptable salt or solvate thereof, for use in the prevention and/or treatment of COVID-19 associated pneumonia in a subject in need thereof.

In one embodiment, COVID-19 associated pneumonia affects both lungs. In one embodiment, COVID-19 associated pneumonia presents on a lung scan (such as computerized tomography (CT) scan) as hazy patches, in particular hazy patches clustering on the outer edges of the lungs. In one embodiment, COVID-19 associated pneumonia presents on a lung scan as radiological finding of ground-glass opacity abnormalities or radiological finding of a mixed pattern (combination of consolidation, ground glass opacity and reticular opacity in the presence of architectural distortion).

In one embodiment, COVID-19 may lead to COVID-19 associated acute respiratory distress syndrome (ARDS) (also referred to as COVID-19 ARDS). Thus, in one embodiment, the present invention relates to a 2-aminoarylthiazole derivative as described herein, in particular masitinib, or a pharmaceutically acceptable salt or solvate thereof, for use in the prevention and/or treatment of COVID-19 associated ARDS in a subject in need thereof.

In one embodiment, ARDS is defined as a form of acute lung injury (ALI) and occurs as a result of a severe pulmonary injury that causes alveolar damage heterogeneously throughout the lung.

In one embodiment, the subject is a male. In one embodiment, the subject is a female.

In one embodiment, the subject is younger than 80, 75, 70, 65 or 60 years of age. In one embodiment, the subject is 80 years old or younger. In one embodiment, the subject is 60 years old or younger. In one embodiment, the subject is older than 40 years of age. In one embodiment, the subject is older than 60, 65, 70 or 75 years of age. In one embodiment, the subject is older than 60, 65, 70 or 75 years of age and younger than 80 years of age. In on embodiment, the subject is 60 years old or older. In one embodiment, the subject is 60 years old or older and younger than 80 years old. In one embodiment, the subject is 80 years of age or older. In one embodiment, the subject is older than 80 years of age. In one embodiment, the subject is living in a nursing home or a long-term care facility.

In one embodiment, the subject is not hospitalized. In one embodiment, the subject is hospitalized. In one embodiment, the subject is hospitalized but does not require admission to intensive care unit (ICU). In one embodiment, the subject is hospitalized and requires admission to intensive care unit (ICU). In one embodiment, the subject does not require oxygen therapy. In one embodiment, the subject requires oxygen therapy. In one embodiment, the subject requires NIV. In one embodiment, the subject requires invasive ventilation, such as intubation and mechanical ventilation.

In one embodiment, the subject to be treated according to the present invention did not receive or is not receiving any other active agent. In one embodiment, the subject to be treated according to the present invention did not receive or is not receiving any other antiviral agent.

Thus, in one embodiment, the 2-aminoarylthiazole derivative as described hereinabove, preferably masitinib, or a pharmaceutically acceptable salt or solvate thereof, is for administration as a first-line treatment. In one embodiment, the 2-aminoarylthiazole derivative as described hereinabove, preferably masitinib, or a pharmaceutically acceptable salt or solvate thereof, is for administration as the sole active agent.

In one embodiment, the subject has interleukin 6 (IL6) plasma levels, in particular interleukin 6 (IL6) plasma levels at baseline, higher than 20 μg/mL. In one embodiment, the subject has interleukin 6 (IL6) plasma levels, in particular interleukin 6 (IL6) plasma levels at baseline, equal or lower than 20 μg/mL.

In one embodiment, the subject is at risk of developing a disease caused by a nidovirus infection or a picornavirus infection, preferably by a coronavirus infection, such as COVID-19 caused by a SARS-CoV-2 infection. In one embodiment, the subject is at risk of developing a severe or critical form of the disease caused by a coronavirus infection, such as COVID-19 caused by a SARS-CoV-2 infection. In one embodiment, the subject is at risk of developing a severe or critical COVID-19 as described hereinabove. In one embodiment, the subject is suffering from COVID-19 and is at risk of developing at least one of the following: pneumonia, acute respiratory distress syndrome (ARDS), sepsis, septic shock, altered consciousness, and/or multi-organ failure.

In one embodiment the subject presents at least one risk factor that may lead to an increased risk of developing a disease caused by a nidovirus infection or a picornavirus infection, preferably by a coronavirus infection, such as COVID-19 caused by a SARS-CoV-2 infection. In one embodiment the subject presents at least one risk factor that may lead to an increased risk of developing a severe or critical form of the disease caused by a coronavirus infection, such as COVID-19 caused by a SARS-CoV-2 infection. In one embodiment the subject presents at least one risk factor that may lead to an increased risk of developing a severe or critical COVID-19 as described hereinabove.

As used herein, “risk factor” refers to a preexisting disease, condition, habit or behavior that may lead to an increased risk of developing a disease caused by a nidovirus infection or a picornavirus infection, preferably by a coronavirus infection, such as COVID-19 caused by a SARS-CoV-2 infection. In one embodiment, “risk factor” refers to a preexisting disease, condition, habit or behavior that may lead to an increased risk of developing a severe or critical form of the disease caused by a coronavirus infection, such as COVID-19 caused by a SARS-CoV-2 infection.

In one embodiment, the subject presents at least one risk factor selected from the group comprising or consisting of active chemotherapy or radical radiotherapy for lung cancer, active smoking, acute kidney injury, asthma, atopy, autoimmune diseases or conditions, auto-inflammatory diseases or conditions, bone marrow or stem cell transplantations in the past 6 months, bronchial hyperreactivity, cancers of the blood or bone marrow (such as leukemia, lymphoma, or myeloma) under any stage of treatment, cardiovascular diseases or conditions, chronic bronchitis, chronic kidney diseases, chronic obstructive pulmonary disease (COPD), chronic passive smoking (also referred to as environmental exposure smoking), cystic fibrosis, diabetes, emphysema, hematological diseases, high blood pressure, immunodeficiency, immunosuppression therapy in particular immunosuppression therapy sufficient to significantly increase the risk of infection, immunotherapy or antibody treatment for cancer, infection with HIV (human immunodeficiency virus), lung cancer, obesity, pregnant women in particular pregnant women who have significant heart disease (whether congenital or acquired), pulmonary hypertension, rare diseases and inborn errors of metabolism that significantly increase the risk of infections (such as severe combined immunodeficiency or homozygous sickle cell), reactive airway disease, recipient of solid organ transplants, severe respiratory conditions, sickle cell disease, solid cancers and targeted cancer treatments that can affect the immune system (such as protein kinase inhibitors or PARP inhibitors).

In one embodiment, the subject is suffering from at least one comorbidity.

As used herein, “comorbidity” refers to a disease or condition coexisting with a nidovirus infection or a picornavirus infection, preferably a coronavirus infection, such as a SARS-CoV-2 infection causing COVID-19, in the subject to be treated according to the present invention. Examples of comorbidities that may coexist with a nidovirus infection or a picornavirus infection, preferably a coronavirus infection, such as a SARS-CoV-2 infection causing COVID-19, in the subject to be treated according to the present invention include, without being limited to, acute kidney injury, asthma, atopy, autoimmune diseases or conditions, auto-inflammatory diseases or conditions, bone marrow or stem cell transplantations in the past 6 months, bronchial hyperreactivity, cancers of the blood or bone marrow (such as leukemia, lymphoma, or myeloma) under any stage of treatment, cardiovascular diseases or conditions, chronic bronchitis, chronic kidney diseases, chronic obstructive pulmonary disease (COPD), cystic fibrosis, diabetes, emphysema, hematological diseases, high blood pressure, immunodeficiency, infection with HIV, lung cancer, obesity, pulmonary hypertension, rare diseases and inborn errors of metabolism that significantly increase the risk of infections (such as severe combined immunodeficiency or homozygous sickle cell), reactive airway disease, recipient of solid organ transplants, severe respiratory conditions, sickle cell disease, and solid cancers.

In one embodiment, the subject presents at least one comorbidity selected from the group comprising or consisting of acute kidney injury, asthma, atopy, autoimmune diseases or conditions, auto-inflammatory diseases or conditions, bone marrow or stem cell transplantations in the past 6 months, bronchial hyperreactivity, cancers of the blood or bone marrow (such as leukemia, lymphoma, or myeloma) under any stage of treatment, cardiovascular diseases or conditions, chronic bronchitis, chronic kidney diseases, chronic obstructive pulmonary disease (COPD), cystic fibrosis, diabetes, emphysema, hematological diseases, high blood pressure, immunodeficiency, infection with HIV, lung cancer, obesity, pulmonary hypertension, rare diseases and inborn errors of metabolism that significantly increase the risk of infections (such as severe combined immunodeficiency or homozygous sickle cell), reactive airway disease, recipient of solid organ transplants, severe respiratory conditions, sickle cell disease, and solid cancers.

In one embodiment, the subject is suffering from sickle cell disease.

In one embodiment, the subject is at least one of the following: an active smoker or a chronic passive smoker (that is to say the subject is exposed to environmental smoking), immunocompromised, pregnant in particular with significant heart disease (whether congenital or acquired), undergoing active chemotherapy or radical radiotherapy for lung cancer, undergoing immunosuppression therapy in particular immunosuppression therapy sufficient to significantly increase the risk of infection, undergoing immunotherapy or antibody treatment for cancer, undergoing targeted cancer treatments that can affect the immune system (such as protein kinase inhibitors or PARP inhibitors).

In the present invention, a 2-aminoarylthiazole derivative refers to a compound characterized by the presence of a thiazolyl group substituted on position 2 (i.e., between the heterocyclic nitrogen and sulfur atoms) by a secondary or tertiary amine, wherein the nitrogen atom of the amine is substituted by at least one aryl group.

According to one embodiment, the aryl group is substituted by an arylamide group (i.e., —NH—CO-aryl).

In one embodiment, the 2-aminoarylthiazole derivative of the invention has the following formula (I):

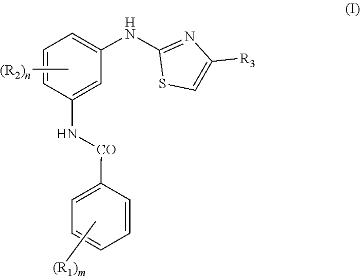

wherein:
R₁ and R₂ are selected independently from hydrogen, halogen, (C₁-C₁₀) alkyl, (C₃-C₁₀) cycloalkyl group, trifluoromethyl, alkoxy, cyano, dialkylamino, a solubilizing group, and (C₁-C₁₀) alkyl substituted by a solubilizing group;
m is 0-5;
n is 0-4;
R₃ is one of the following:
   (i) an aryl group (such as phenyl), the aryl group being optionally substituted by one or more substituents such as halogen, (C₁-C₁₀) alkyl group, trifluoromethyl, cyano and alkoxy;
   (ii) a heteroaryl group (such as 2, 3, or 4-pyridyl group), the heteroaryl group being optionally substituted by one or more substituents such as halogen, (C₁-C₁₀) alkyl group, trifluoromethyl and alkoxy;
   (iii) a five-membered ring aromatic heterocyclic group (such as, for example, 2-thienyl, 3-thienyl, 2-thiazolyl, 4-thiazolyl, 5-thiazolyl), the aromatic heterocyclic group being optionally substituted by one or more substituents such as halogen, (C₁-C₁₀) alkyl group, trifluoromethyl, and alkoxy.

In one embodiment, R₁ and R₂ of formula (I) are selected independently from hydrogen, halogen, (C₁-C₁₀) alkyl, (C₃-C₁₀) cycloalkyl group, trifluoromethyl, alkoxy, cyano, dialkylamino, and a solubilizing group.

Thus, in one embodiment, the 2-aminoarylthiazole derivative of the invention or a pharmaceutically acceptable salt or solvate thereof is a 2-aminoarylthiazole derivative of formula (I) as described above or a pharmaceutically acceptable salt or solvate thereof.

In one embodiment, the 2-aminoarylthiazole derivative of the invention has the following formula (II):

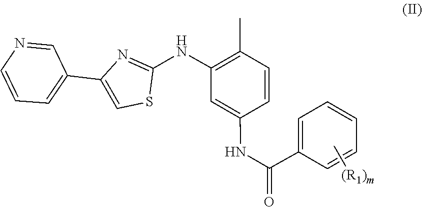

wherein:
R₁ is selected independently from hydrogen, halogen, (C₁-C₁₀) alkyl, (C₃-C₁₀) cycloalkyl group, trifluoromethyl, alkoxy, amino, alkylamino, dialkylamino, a solubilizing group, and (C₁-C₁₀) alkyl substituted by a solubilizing group; and
m is 0-5.

In one embodiment, R₁ of formula (II) is selected independently from hydrogen, halogen, (C₁-C₁₀) alkyl, (C₃-C₁₀) cycloalkyl group, trifluoromethyl, alkoxy, amino, alkylamino, dialkylamino, and a solubilizing group.

In one embodiment, R₁ of formula (II) is a solubilizing group. In one embodiment, R₁ of formula (II) is (C₁-C₁₀) alkyl substituted by a solubilizing group.

In one embodiment, R₁ of formula (II) is (C1-C10) alkyl-(C2-C11) heterocycloalkyl-(C1-C10) alkyl-. In one embodiment, R₁ of formula (II) is (C₁-C₄) alkyl-(C₂-C₁₁) heterocycloalkyl-(C₁-C₁₀) alkyl-, preferably (C₁-C₂) alkyl-(C₂-C₁₁) heterocycloalkyl-(C₁-C₁₀) alkyl-. In one embodiment, R₁ of formula (II) is (C₁-C₁₀) alkyl-(C₂-C₁₁) heterocycloalkyl-(C₁-C₄) alkyl-, preferably (C₁-C₁₀) alkyl-(C₂-C₁₁) heterocycloalkyl-(C₁-C₂) alkyl-. In one embodiment, R₁ of formula (II) is (C₁-C₁₀) alkyl-(C₂-C₆) heterocycloalkyl-(C₁-C₁₀) alkyl-, preferably (C₁-C₁₀) alkyl-(C₄) heterocycloalkyl-(C₁-C₁₀) alkyl-. In one embodiment, R₁ of formula (II) is (C₁-C₄) alkyl-(C₂-C₆) heterocycloalkyl-(C₁-C₄) alkyl-, preferably (C₁-C₂) alkyl-(C₄) heterocycloalkyl-(C₁-C₂) alkyl-. In one embodiment, R₁ of formula (II) is (C₁-C₄) alkyl-piperazinyl-(C₁-C₄) alkyl-, preferably (C₁-C₂) alkyl-piperazinyl-(C₁-C₂) alkyl-. In one embodiment, R₁ of formula (II) is methylpiperazinyl-(C₁-C₂) alkyl-, preferably methylpiperazinyl-methyl-, more preferably 4-methylpiperazinyl-methyl-.

Thus, in one embodiment, the 2-aminoarylthiazole derivative of the invention or a pharmaceutically acceptable salt or solvate thereof is a 2-aminoarylthiazole derivative of formula (II) as described above or a pharmaceutically acceptable salt or solvate thereof.

As used herein, the term “aryl group” refers to a polyunsaturated, aromatic hydrocarbyl group having a single aromatic ring (i.e., phenyl) or multiple aromatic rings fused together (e.g., naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Examples of suitable aryl groups include, without being limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C₆) aryl”.

As used herein, the term “alkyl group” refers to a saturated straight chain or branched non-cyclic hydrocarbon having from 1 to 10 carbon atoms, preferably from 1 to 6 carbon atoms. Representative saturated straight chain alkyls include, without being limited to, methyl, ethyl, n-propyl, n-butyl, n-pentyl, n-hexyl, n-heptyl, n-octyl, n-nonyl and n-decyl. Saturated branched alkyls include, without being limited to, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl. Alkyl groups included in compounds of the present invention may be optionally substituted with one or more substituents.

As used herein, the term “alkoxy” refers to an alkyl group which is attached to another moiety by an oxygen atom. Examples of alkoxy groups include, without being limited to, methoxy, isopropoxy, ethoxy, tert-butoxy. Alkoxy groups may be optionally substituted with one or more substituents.

As used herein, the term “cycloalkyl” refers to a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

As used herein, the term “halogen” refers to —F, —Cl, —Br or —I.

As used herein, the term “heteroaryl” refers to a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include, without being limited to, pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

As used herein, the term “heterocycle” refers collectively to heterocycloalkyl groups and heteroaryl groups.

As used herein, the term “heterocycloalkyl” refers to a monocyclic or polycyclic group having at least one heteroatom selected from O, N, or S, and which has 2-11 carbon atoms, which may be saturated or unsaturated, but is not aromatic. Examples of heterocycloalkyl groups include, without being limited to, piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein, the term “substituent” or “substituted” means that a hydrogen radical on a compound or group is replaced with any desired group that is substantially stable to reaction conditions in an unprotected form or when protected using a protecting group. Examples of preferred substituents include, without being limited to, halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain embodiments, the term “substituent” or the adjective “substituted” refers to a solubilizing group.

As used herein, the term “solubilizing group” refers to any group which can be substantially ionized and that enables the compound to be soluble in a desired solvent, such as, for example, water or water-containing solvent (“water-solubilizing group”). Furthermore, the solubilizing group can be one that increases the compound or complex's lipophilicity. In one embodiment, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. In one embodiment, the solubilizing group is one of the following:
an alkyl, cycloalkyl, aryl, heteroaryl group comprising either at least one nitrogen or oxygen heteroatom and/or which group is substituted by at least one amino group or oxo group (including, without being limited to, 2-oxopiperazinyl, 2-oxopiperidinyl, 2-oxopyrrolidinyl, 4-piperidonyl, hydantoinyl, valerolactamyl, oxiranyl, oxetanyl, tetrahydropyranyl, morpholinyl, 1,3-dioxolane, tetrahydrofuranyl and dihydrofuranyl-2-one);
an amino group which may be a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl);
one of the structures a) to i) shown below, wherein the wavy line and the arrow line correspond to the point of attachment to the core structure of the 2-aminoarylthiazole derivative of the invention, for example of formula (I) or (II):

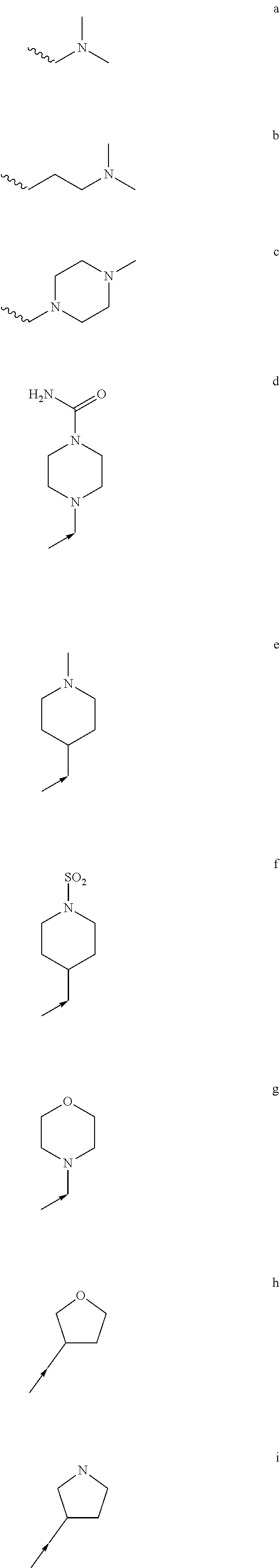

In one embodiment, the solubilizing group is a saturated cyclic amino group (including, without being limited to, piperidinyl, piperazinyl and pyrrolidinyl) which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl (including, without being limited to, methyl-piperidinyl, methyl-piperazinyl and methyl-pyrrolidinyl).

In one embodiment, the solubilizing group is structure c) shown above, wherein the wavy line corresponds to the point of attachment to the core structure of the 2-aminoarylthiazole derivative of the invention, for example of formula (I) or (II).

As used herein, “pharmaceutically acceptable salt” refers to a salt of a free acid or a free base which is not biologically undesirable and is generally prepared by reacting the free base with a suitable organic or inorganic acid or by reacting the free acid with a suitable organic or inorganic base. Suitable acid addition salts are formed from acids that form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, napsylate, nicotinate, nitrate, orotate, oxalate, palmitate, pamoate, phosphate/hydrogen, phosphate/dihydrogen, phosphate, pyroglutamate, saccharate, stearate, succinate, tannate, tartrate, tosylate, trifluoroacetate and xinofoate salts. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine, 2 (diethylamino)ethanol, ethanolamine, morpholine, 4 (2 hydroxyethyl)morpholine and zinc salts. Hemi salts of acids and bases may also be formed, e.g., hemi sulphate and hemi calcium salts.

In one embodiment, pharmaceutically acceptable salts are pharmaceutically acceptable acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

In one embodiment, the pharmaceutically acceptable salt of the 2-aminoarylthiazole derivative of the invention is mesilate.

Unless otherwise indicated, the term “mesilate” is used herein to refer to a salt of methanesulfonic acid with a named pharmaceutical substance (such as compounds of formula (I) or (II)). Use of mesilate rather than mesylate is in compliance with the INNM (International nonproprietary names modified) issued by WHO (e.g., World Health Organization (February 2006). International Nonproprietary Names Modified. INN Working Document 05.167/3. WHO).

As used herein, “pharmaceutically acceptable solvate” refers to a molecular complex comprising the 2-aminoarylthiazole derivative of the invention and stoichiometric or sub-stoichiometric amounts of one or more pharmaceutically acceptable solvent molecules such as ethanol. The term “hydrate” refers to when said solvent is water.

According to one embodiment, the 2-aminoarylthiazole derivative of the invention or a pharmaceutically acceptable salt or solvate thereof is masitinib or a pharmaceutically acceptable salt or solvate thereof.

The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5:

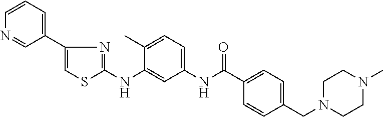 [SMILES: Cc1ccc(NC(=O)c2ccc(CN3CCN(C)CC3)cc2)cc1Nc1nc(-c2cccnc2)cs1]

Masitinib was first described in U.S. Pat. No. 7,423,055 and EP 1 525 200 Bl.

According to one embodiment, the 2-aminoarylthiazole derivative of the invention, or a pharmaceutically acceptable salt or solvate thereof, is masitinib mesilate. Thus, in one embodiment, the pharmaceutically acceptable salt of masitinib as described hereinabove is masitinib mesilate. As mentioned hereinabove, in other words, the pharmaceutically acceptable salt of masitinib is the methanesulfonic acid salt of masitinib.

A detailed procedure for the synthesis of masitinib mesilate is given in WO 2008/098949.

In one embodiment, “masitinib mesilate” refers to the orally bioavailable mesylate salt of masitinib—CAS 1048007-93-7 (MsOH); C28H30N60S·CH3SO3H; MW 594.76:

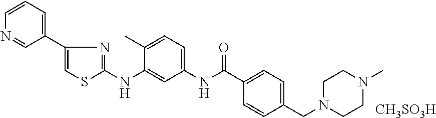

According to one embodiment, the 2-aminoarylthiazole derivative as described hereinabove, preferably masitinib, or a pharmaceutically acceptable salt or solvate thereof, is for administration as the sole active agent, i.e., the sole agent exhibiting a biological or pharmacological activity. Thus, according to one embodiment, the 2-aminoarylthiazole derivative as described hereinabove, preferably masitinib, or a pharmaceutically acceptable salt or solvate thereof, is not for administration with another active agent, such as another antiviral agent. In one embodiment, the 2-aminoarylthiazole derivative as described hereinabove, preferably masitinib, or a pharmaceutically acceptable salt or solvate thereof, is not for administration with another antiviral agent.

According to one embodiment, the 2-aminoarylthiazole derivative as described hereinabove, preferably masitinib, or a pharmaceutically acceptable salt or solvate thereof, is for administration at a therapeutically effective dose.

In one embodiment, the 2-aminoarylthiazole derivative as described hereinabove, preferably masitinib, or a pharmaceutically acceptable salt or solvate thereof, is for administration at a dose of at least about 0.01 mg/kg/day (mg per kilo body weight per day), preferably at least about 0.1 mg/kg/day, more preferably at least about 1 mg/kg/day. In one embodiment, the 2-aminoarylthiazole derivative as described hereinabove, preferably masitinib, or a pharmaceutically acceptable salt or solvate thereof, is for administration at a dose ranging from about 1 mg/kg/day to about 500 mg/kg/day, preferably at a dose ranging from about 1 mg/kg/day to about 200 mg/kg/day.

In one embodiment, the 2-aminoarylthiazole derivative as described hereinabove, preferably masitinib, or a pharmaceutically acceptable salt or solvate thereof, is for administration at a dose ranging from about 1 to about 12 mg/kg/day (mg per kilo body weight per day). In one embodiment, the 2-aminoarylthiazole derivative as described hereinabove, preferably masitinib, or a pharmaceutically acceptable salt or solvate thereof, is for administration at a dose ranging from about 1.5 to about 7.5 mg/kg/day. In one embodiment, the 2-aminoarylthiazole derivative as described hereinabove, preferably masitinib, or a pharmaceutically acceptable salt or solvate thereof, is for administration at a dose ranging from about 3 to about 12 mg/kg/day, preferably from about 3 to about 6 mg/kg/day, more preferably from about 3 to about 4.5 mg/kg/day.

In one embodiment, the 2-aminoarylthiazole derivative as described hereinabove, preferably masitinib, or a pharmaceutically acceptable salt or solvate thereof, is for administration at a dose of about 1, 2, 3, 4, 5, 6, 7, 8, 9, 10, 11 or 12 mg/kg/day. In one embodiment, the 2-aminoarylthiazole derivative as described hereinabove, preferably masitinib, or a pharmaceutically acceptable salt or solvate thereof, is for administration at a dose of about 1.5, 3, 4.5, 6, 7.5, 9, 10.5 or 12 mg/kg/day.

In one embodiment, the 2-aminoarylthiazole derivative as described hereinabove, preferably masitinib, or a pharmaceutically acceptable salt or solvate thereof, is for administration at a dose of about 3, 4.5 or 6 mg/kg/day, preferably at a dose of about 3 mg/kg/day or of about 4.5 mg/kg/day.

In one embodiment, the 2-aminoarylthiazole derivative as described hereinabove, preferably masitinib, or a pharmaceutically acceptable salt or solvate thereof, is for administration at a dose as described hereinabove for at least 1, 2, 3, 4, 5, 6, 7, 8, 9 or 10 day(s).

In one embodiment, the 2-aminoarylthiazole derivative as described hereinabove, preferably masitinib, or a pharmaceutically acceptable salt or solvate thereof, can be dose escalated by increments of about 1.5 mg/kg/day to reach a maximum of about 12 mg/kg/day. Each dose escalation is subjected to toxicity controls with an absence of any toxicity events permitting dose escalation to occur.

In one embodiment, the dose escalation of the 2-aminoarylthiazole derivative, or a pharmaceutically acceptable salt or solvate thereof, occurs at any time-point after at least 1 day after the administration of the initial dose; for example, after 1, 2, 3, 4, 5, 6, or 7 day(s), preferably after 4 days, more preferably after 2 days. In one embodiment, the dose escalation of the 2-aminoarylthiazole derivative, or a pharmaceutically acceptable salt or solvate thereof, occurs at any time-point after at least 1 week after the administration of the initial dose; for example, after 1 week, 2 weeks, 3 weeks, or 4 weeks after the administration of the initial dose, preferably after 1 week. Each dose escalation is subjected to toxicity controls. Example of a toxicity control includes assessing that, during the previous 2-day, 4-day or 1-week treatment period at a constant dose of study treatment, no suspected severe adverse event was reported, no suspected adverse event led to treatment interruption, and/or no suspected adverse event is ongoing at the time of the dose increase, regardless of its severity.

In one embodiment, the 2-aminoarylthiazole derivative as described hereinabove, preferably masitinib, or a pharmaceutically acceptable salt or solvate thereof, is for administration at an initial dose of about 3 mg/kg/day during at least 1, 2, 3, 4, 5, 6, or 7 day(s), preferably during at least 4 days, more preferably during at least 2 days, then at a dose of about 4.5 mg/kg/day thereafter, preferably during at least 1, 2, 3, 4, 5, or 6 day(s). In one embodiment, the 2-aminoarylthiazole derivative as described hereinabove, preferably masitinib, or a pharmaceutically acceptable salt or solvate thereof, is for administration at an initial dose of about 3 mg/kg/day during at least 1 week, at least 2 weeks or at least 3 weeks, then at a dose of about 4.5 mg/kg/day thereafter, preferably during at least 1, 2, 3, 4, 5, or 6 day(s). In one embodiment, the 2-aminoarylthiazole derivative as described hereinabove, preferably masitinib, or a pharmaceutically acceptable salt or solvate thereof, is for administration at an initial dose of about 3 mg/kg/day during at least 1, 2, 3, 4, 5, 6, or 7 day(s), preferably during at least 4 days, more preferably during at least 2 days, then at a dose of about 4.5 mg/kg/day during at least 1, 2, 3, 4, 5, 6, or 7 day(s), preferably during at least 4 days, more preferably during at least 2 days, then at a dose of about 6 mg/kg/day thereafter, preferably during at least 1, 2, 3, 4, 5, or 6 day(s). In one embodiment, the 2-aminoarylthiazole derivative as described hereinabove, preferably masitinib, or a pharmaceutically acceptable salt or solvate thereof, is for administration at an initial dose of about 3 mg/kg/day during at least 1 week, at least 2 weeks or at least 3 weeks, then at a dose of about 4.5 mg/kg/day during at least 1 week, at least 2 weeks or at least 3 weeks, then at a dose of about 6 mg/kg/day thereafter, preferably during at least 1, 2, 3, 4, 5, or 6 day(s).

According to one embodiment, any dose indicated herein refers to the amount of active ingredient (also referred to as active agent) as such, not to its pharmaceutically acceptable salt or solvate form. Thus, compositional variations of a pharmaceutically acceptable salt or solvate of the 2-aminoarylthiazole derivative of the invention, in particular masitinib, will not impact the dose to be administered.

According to one embodiment, the 2-aminoarylthiazole derivative as described hereinabove, preferably masitinib, or a pharmaceutically acceptable salt or solvate thereof, may be administered orally, intravenously, parenterally, topically, by inhalation spray, rectally, nasally, or buccally. In one embodiment, the 2-aminoarylthiazole derivative as described hereinabove, preferably masitinib, or a pharmaceutically acceptable salt or solvate thereof, may be administered as an oral, sublingual, transdermal, subcutaneous, topical, for absorption through epithelial or mucocutaneous linings, intravenous, intranasal, intraarterial, intramuscular, intraperitoneal, intrathecal, rectal, vaginal, or aerosol formulation.

Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The active agent may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethyleneglycol (e.g., PEG400), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which may be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof. The parenteral formulations will typically contain from about 0.5 to about 25% by weight of the active agent, i.e., a 2-aminoarylthiazole derivative as described hereinabove, preferably masitinib, or a pharmaceutically acceptable salt or solvate thereof, in solution. Suitable preservatives and buffers may be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets of the kind previously described.

The active agent, i.e., a 2-aminoarylthiazole derivative as described hereinabove, preferably masitinib, or a pharmaceutically acceptable salt or solvate thereof, may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier may be a liquid, solid or semi-solid. In embodiments, the composition is an aqueous solution, such as a mouthwash. Alternatively, the composition may be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions may be produced as solids, such as powders or granules. The solids may be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin, and silicone-based materials.

The active agent, i.e., a 2-aminoarylthiazole derivative as described hereinabove, preferably masitinib, or a pharmaceutically acceptable salt or solvate thereof, may be made into aerosol formulations to be administered via inhalation. These aerosol formulations may be placed into pressurized acceptable propellants. Suitable propellants include, e.g., a fluorinated hydrocarbon (e.g., trichloromonofluoromethane, dichlorodifluoromethane, chlorodifluoromethane, chlorodifluoroethane, dichlorotetrafluoroethane, heptafluoropropane, tetrafluoroethane, difluoroethane), a hydrocarbon (e.g., propane, butane, isobutane), or a compressed gas (e.g., nitrogen, nitrous oxide, carbon dioxide). They also may be formulated as pharmaceuticals for non-pressured preparations, such as in a nebulizer or an atomizer.

In one embodiment, the 2-aminoarylthiazole derivative as described hereinabove, preferably masitinib, or a pharmaceutically acceptable salt or solvate thereof, is for oral administration.

In one embodiment, the 2-aminoarylthiazole derivative as described hereinabove, preferably masitinib, or a pharmaceutically acceptable salt or solvate thereof, is for administration at least once a day, preferably twice a day.

In one embodiment, the 2-aminoarylthiazole derivative as described hereinabove, preferably masitinib, or a pharmaceutically acceptable salt or solvate thereof, is for administration for a period of at least 1, 2, 3, 4, 5 or 6 weeks, preferably of at least 2 weeks. In one embodiment, the 2-aminoarylthiazole derivative as described hereinabove, preferably masitinib, or a pharmaceutically acceptable salt or solvate thereof, is for administration for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, preferably of at least 15 days.

In one embodiment, the 2-aminoarylthiazole derivative as described hereinabove, preferably masitinib, or a pharmaceutically acceptable salt or solvate thereof, is in a form adapted for oral administration. Examples of forms adapted for oral administration include, without being limited to, liquid, paste or solid compositions, and more particularly tablets, capsules, pills, liquids, gels, syrups, slurries, and suspensions.

In one embodiment, the 2-aminoarylthiazole derivative as described hereinabove, preferably masitinib, or a pharmaceutically acceptable salt or solvate thereof, is for administration as a tablet, preferably as a 100 mg or a 200 mg tablet.

According to one embodiment, the 2-aminoarylthiazole derivative as described hereinabove, preferably masitinib, or a pharmaceutically acceptable salt or solvate thereof, is for administration in combination with quercetin flavonols, such as isoquercetin, quercetin, or quercetin-3-O-β-D-glucuronide.

In one embodiment, the 2-aminoarylthiazole derivative as described hereinabove, preferably masitinib, or a pharmaceutically acceptable salt or solvate thereof, is for administration in combination with isoquercetin or quercetin, preferably with isoquercetin.

Isoquercetin (CAS number 482-35-9) is also known as quercetin 3-O-glucopyranoside, quercetin-3-O-glucoside, isoquercitroside, isotrifoliin, trifolin, trifolin A or isoquercitrin. Its molecular formula is C21H20O12 and its IUPAC (International Union of Pure and Applied Chemistry) name is 2-(3,4-dihydroxyphenyl)-5,7-dihydroxy-3-[(2S,3R,4S,5S,6R)-3,4,5-trihydroxy-6-(hydroxymethyl)oxan-2-yl]oxychromen-4-one.

Isoquercetin has the following formula:

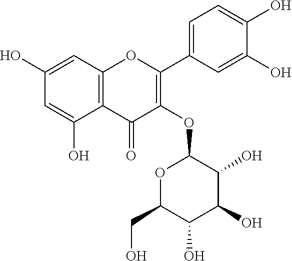 [SMILES: O=c1c(O[C@@H]2O[C@H](CO)[C@@H](O)[C@H](O)[C@H]2O)c(-c2ccc(O)c(O)c2)oc2cc(O)cc(O)c12]

As used herein, the term “isoquercetin” encompasses the crystalline solid form, any prodrugs, pharmaceutically acceptable salts, hydrates and solvates thereof.

Isoquercetin is a flavonol belonging to a broad group of pigmented substances of plant origin known as flavonoids. Flavonoids are the largest group of naturally occurring polyphenolic compounds with diverse biological activities. Isoquercetin is an orally bioavailable derivative of quercetin.

Quercetin (CAS number 117-39-5) is also known as sophoretin, meletin, xanthaurine, quercetol, quercitin, quertine, 2-(3,4-dihydroxyphenyl)-3,5,7-trihydroxy-4H-1-benzopyran-4-one, 3,3′,4′,5,7-pentahydroxyflavone or 3,5,7,3′,4′-Pentahydroxyflavone. Its molecular formula is C15H10O7 and its IUPAC name is 2-(3,4-dihydroxyphenyl)-3,5,7-trihydroxychromen-4-one.

Quercetin has the following formula:

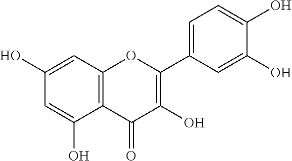 [SMILES: O=c1c(O)c(-c2ccc(O)c(O)c2)oc2cc(O)cc(O)c12]

As used herein, the term “quercetin” encompasses the crystalline solid form, any prodrugs, pharmaceutically acceptable salts, hydrates and solvates thereof.

Quercetin is an abundant polyphenolic flavonoid that has been isolated from a variety of fruits and vegetables and has diverse biological activities.

An object of the invention is thus a 2-aminoarylthiazole derivative as described hereinabove, preferably masitinib, or a pharmaceutically acceptable salt or solvate thereof, in combination with isoquercetin or quercetin, preferably with isoquercetin, for use in the treatment of a nidovirus infection or a picornavirus infection as described hereinabove, preferably a coronavirus infection, in particular a SARS-CoV-2 infection causing COVID-19, in a subject in need thereof as described hereinabove.

In one embodiment, the present invention relates to a 2-aminoarylthiazole derivative as described hereinabove, preferably masitinib, or a pharmaceutically acceptable salt or solvate thereof, in combination with isoquercetin for use in the treatment of a coronavirus infection, in particular of a SARS-CoV-2 infection causing COVID-19, in a subject in need thereof as described hereinabove.

In one embodiment, the present invention relates to a 2-aminoarylthiazole derivative as described hereinabove, preferably masitinib, or a pharmaceutically acceptable salt or solvate thereof, in combination with isoquercetin for use in the prevention and/or treatment of COVID-19 in a subject in need thereof as described hereinabove.

In one embodiment, the 2-aminoarylthiazole derivative as described hereinabove, preferably masitinib, or a pharmaceutically acceptable salt or solvate thereof, is thus for simultaneous, separate or sequential administration with isoquercetin or quercetin.

In one embodiment, the 2-aminoarylthiazole derivative as described hereinabove, preferably masitinib, or a pharmaceutically acceptable salt or solvate thereof, is for combined administration with isoquercetin or quercetin, for example in a combined preparation, pharmaceutical composition or medicament.

Another object of the invention is a combination of a 2-aminoarylthiazole derivative as described hereinabove, preferably masitinib, or a pharmaceutically acceptable salt or solvate thereof, with isoquercetin or quercetin, preferably with isoquercetin, for use in the treatment of a nidovirus infection or a picornavirus infection as described hereinabove, preferably a coronavirus infection, in particular a SARS-CoV-2 infection causing COVID-19, in a subject in need thereof as described hereinabove.

In one embodiment, the present invention relates to a combination of masitinib, or a pharmaceutically acceptable salt or solvate thereof, with isoquercetin for use in the treatment of a coronavirus infection, in particular of a SARS-CoV-2 infection causing COVID-19, in a subject in need thereof as described hereinabove.

In one embodiment, the present invention relates to a combination of masitinib, or a pharmaceutically acceptable salt or solvate thereof, with isoquercetin for use in the prevention and/or treatment of COVID-19 in a subject in need thereof as described hereinabove.

In one embodiment, the combination of the invention is a simultaneous, separate or sequential combination. In one embodiment, the combination of the invention is a combined preparation, pharmaceutical composition or medicament.

In one embodiment, isoquercetin as described hereinabove is for administration at a dose ranging from about 0.25 g/day to about 5 g/day, preferably ranging from about 0.5 g/day to about 2.5 g/day, more preferably ranging from about 1 g/day to about 2 g/day.

In one embodiment, isoquercetin as described hereinabove is for administration at a dose ranging from about 0.4 g/day to about 2 g/day.

In one embodiment, isoquercetin as described hereinabove is for administration at a dose of about 0.5, 1, 1.5, 2, 2.5, 3, 3.5, 4, 4.5, or 5 g/day. In one embodiment, isoquercetin as described hereinabove is for administration at a dose of about 1 g/day.

In one embodiment, isoquercetin as described hereinabove can be administered at doses reduced at regular intervals by increments of about 0.5 g/day.

In one embodiment, the dose reduction of isoquercetin as described hereinabove occurs at any time-point after at least 7 days after the administration of the initial dose and prior to 28 days after the administration of the initial dose; for example, 7 days, 14 days, or 21 days after the administration of the initial dose.

In one embodiment, isoquercetin as described hereinabove is for administration at an initial dose of about 2 g/day during at least 7 days, 14 days or 21 days, then at a dose of about 1.5 g/day thereafter.

In one embodiment, isoquercetin as described hereinabove is for administration at an initial dose of about 2 g/day during at least 7 days, then at a dose of about 1.5 g/day during at least 7 days, and at a dose of about 1 g/day thereafter.

According to one embodiment, isoquercetin as described hereinabove may be administered orally, intravenously, parenterally, topically, by inhalation spray, rectally, nasally, or buccally.

In one embodiment, isoquercetin as described hereinabove is for oral administration.

In one embodiment, isoquercetin as described hereinabove is for administration at least once a day, preferably twice a day.

In one embodiment, isoquercetin as described hereinabove is for administration for a period of at least 1, 2, 3, 4, 5 or 6 weeks, preferably of at least 2 weeks. In one embodiment, isoquercetin as described hereinabove is for administration for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, preferably of at least for 15 days.

In one embodiment, isoquercetin as described hereinabove is in a form adapted for oral administration. Examples of forms adapted for oral administration are indicated hereinabove.

In one embodiment, isoquercetin as described hereinabove is for administration as a capsule, preferably as a 250 mg capsule.

Another object of the invention is a kit-of-parts comprising a first part comprising a 2-aminoarylthiazole derivative as described hereinabove, preferably masitinib, or a pharmaceutically acceptable salt or solvate thereof, and a second part comprising isoquercetin or quercetin, preferably isoquercetin, as described hereinabove.

In one embodiment, the kit-of-parts of the invention comprises a first part comprising a masitinib, or a pharmaceutically acceptable salt or solvate thereof, and a second part comprising isoquercetin.

According to one embodiment, the 2-aminoarylthiazole derivative of the invention, preferably masitinib, or a pharmaceutically acceptable salt or solvate thereof, optionally with isoquercetin or quercetin, is for administration with at least one further pharmaceutically active agent.

In one embodiment, the 2-aminoarylthiazole derivative as described hereinabove, or a pharmaceutically acceptable salt or solvate thereof, in combination with isoquercetin or quercetin, preferably with isoquercetin, is for administration with at least one further pharmaceutically active agent.

In one embodiment, the combination of a 2-aminoarylthiazole derivative, or a pharmaceutically acceptable salt or solvate thereof, with isoquercetin or quercetin, preferably with isoquercetin, as described hereinabove is for administration with at least one further pharmaceutically active agent.

According to the present invention, the 2-aminoarylthiazole derivative, preferably masitinib, or a pharmaceutically acceptable salt or solvate thereof, optionally with isoquercetin or quercetin, may be administered simultaneously, separately or sequentially with said at least one further pharmaceutically active agent.

In one embodiment, the 2-aminoarylthiazole derivative, preferably masitinib, or a pharmaceutically acceptable salt or solvate thereof, as described hereinabove, optionally with isoquercetin or quercetin, is for administration in combination with said at least one further pharmaceutically active agent, preferably in a combined preparation, pharmaceutical composition or medicament.

Examples of further pharmaceutically active agents that may be administered to a subject with a nidovirus infection or a picornavirus infection as described hereinabove, preferably a coronavirus infection, in particular a SARS-CoV-2 infection causing COVID-19, include, without being limited to, antiviral agents, anti-interleukin 6 (anti-IL6) agents, protease inhibitors, Janus-associated kinase (JAK) inhibitors, and other agents such as BXT-25, brilacidin, dehydroandrographolide succinate, APNO1, fingolimod, methylprednisolone, thalidomide, bevacizumab, sildenafil citrate, interferon, or carrimycin.

In one embodiment, the at least one further pharmaceutically active agent is selected from the group comprising or consisting of antiviral agents; anti-interleukin 6 (anti-IL6) agents; protease inhibitors; JAK inhibitors; other agents such as BXT-25, brilacidin, dehydroandrographolide succinate, APNO1, fingolimod, methylprednisolone, thalidomide, bevacizumab, sildenafil citrate, interferon, carrimycin, angiotensin receptor-blocker (ARB), angiotensin-converting-enzyme inhibitors (ACE-I), losartan, or CD24Fc; and any mixes thereof.

In one embodiment, the at least one further pharmaceutically active agent is selected from the group comprising or consisting of antiviral agents; anti-interleukin 6 (anti-IL6) agents; protease inhibitors; JAK inhibitors; other agents such as BXT-25, brilacidin, dehydroandrographolide succinate, APN01, fingolimod, methylprednisolone, thalidomide, bevacizumab, sildenafil citrate, interferon, carrimycin, and any mixes thereof.

In one embodiment, the at least one further pharmaceutically active agent is selected from the group comprising or consisting of remdesivir, a combination of lopinavir and ritonavir (lopinavir/ritonavir) with or without interferon (such as interferon beta-1a (IFN-β-1a), interferon beta-1b (IFN-β-1b) and peginterferon beta-1a), a combination of darunavir and cobicistat (darunavir/cobicistat), oseltamivir, favipiravir hydroxychloroquine, chloroquine, tocilizumab, sarilumab, baricitinib, fingolimod, methylprednisolone, thalidomide, bevacizumab, sildenafil citrate, interferon (such as interferon beta-1a (IFN-β-1a), interferon beta-1b (IFN-β-1b) and peginterferon beta-1a), carrimycin, angiotensin receptor-blocker (ARB), angiotensin-converting-enzyme inhibitors (ACE-I), losartan, CD24Fc, and any mixes thereof.

In one embodiment, the at least one further pharmaceutically active agent is selected from the group comprising or consisting of remdesivir, a combination of lopinavir and ritonavir (lopinavir/ritonavir) with or without interferon (such as interferon beta-1a (IFN-β-1a), interferon beta-1b (IFN-β-1b) and peginterferon beta-1a), hydroxychloroquine, anti-IL6 agents (such as tocilizumab, siltuximab, sarilumab, sirukumab, clazakizumab, or olokizumab) and any mixes thereof.

In one embodiment, the at least one further pharmaceutically active agent is selected from the group comprising or consisting of remdesivir, a combination of lopinavir and ritonavir (lopinavir/ritonavir) with or without interferon (such as interferon beta-1a (IFN-β-1a), interferon beta-1b (IFN-β-1b) and peginterferon beta-1a), hydroxychloroquine and any mixes thereof.

In one embodiment, the at least one further pharmaceutically active agent is an antiviral agent. Example of antiviral agents that may be administered to a subject with a nidovirus infection or a picornavirus infection as described hereinabove, preferably a coronavirus infection, in particular a SARS-CoV-2 infection causing COVID-19, include, without being limited to, remdesivir, a combination of lopinavir and ritonavir (lopinavir/ritonavir), chloroquine, hydroxychloroquine, ribavirin, oseltamivir, beclabuvir, saquinavir, umifenovir, favipiravir, leronlimab, a combination of darunavir and cobicistat (darunavir/cobicistat), galidesivir and fabiravir.

In one embodiment, the at least one further pharmaceutically active agent is an antiviral agent selected from the group comprising or consisting of remdesivir, a combination of lopinavir and ritonavir (lopinavir/ritonavir), chloroquine, hydroxychloroquine, ribavirin, oseltamivir, beclabuvir, saquinavir, umifenovir, favipiravir, leronlimab, a combination of darunavir and cobicistat (darunavir/cobicistat), galidesivir, fabiravir and any mixes thereof.

In one embodiment, the at least one further pharmaceutically active agent is an antiviral agent selected from the group comprising or consisting of remdesivir, a combination of lopinavir and ritonavir (lopinavir/ritonavir), chloroquine, hydroxychloroquine, oseltamivir, favipiravir, a combination of darunavir and cobicistat (darunavir/cobicistat), galidesivir, fabiravir and any mixes thereof.

In one embodiment, the at least one further pharmaceutically active agent is an antiviral agent selected from the group comprising or consisting of remdesivir, a combination of lopinavir and ritonavir (lopinavir/ritonavir), chloroquine, hydroxychloroquine, and any mixes thereof.

In one embodiment, the at least one further pharmaceutically active agent is an antiviral agent selected from the group comprising or consisting of remdesivir, a combination of lopinavir and ritonavir (lopinavir/ritonavir), hydroxychloroquine, and any mixes thereof.

In one embodiment, the at least one further pharmaceutically active agent is an antiviral agent selected from the group comprising or consisting of remdesivir, hydroxychloroquine, and any mixes thereof.

In one embodiment, the at least one further pharmaceutically active agent is an anti-IL6 agent. Example of anti-IL6 agents that may be administered to a subject with a nidovirus infection or a picornavirus infection as described hereinabove, preferably a coronavirus infection, in particular a SARS-CoV-2 infection causing COVID-19, include, without being limited to, tocilizumab, siltuximab, sarilumab, sirukumab, clazakizumab, and olokizumab.

In one embodiment, the at least one further pharmaceutically active agent is an anti-IL6 agent selected from the group comprising or consisting of tocilizumab, siltuximab, sarilumab, sirukumab, clazakizumab, olokizumab, and any mixes thereof.

In one embodiment, the at least one further pharmaceutically active agent is tocilizumab.

In one embodiment, the at least one further pharmaceutically active agent is a protease inhibitor. Example of protease inhibitors that may be administered to a subject with a nidovirus infection or a picornavirus infection as described hereinabove, preferably a coronavirus infection, in particular a SARS-CoV-2 infection causing COVID-19, include, without being limited to, simeprevir and camostat mesylate

In one embodiment, the at least one further pharmaceutically active agent is a protease inhibitor selected from the group comprising or consisting of simeprevir, camostat mesylate, and any mixes thereof.

In one embodiment, the at least one further pharmaceutically active agent is a JAK inhibitor. Example of JAK inhibitors that may be administered to a subject with a nidovirus infection or a picornavirus infection as described hereinabove, preferably a coronavirus infection, in particular a SARS-CoV-2 infection causing COVID-19, include, without being limited to, baricitinib, fedratinib and ruxolitinib.

In one embodiment, the at least one further pharmaceutically active agent is a JAK inhibitor selected from the group comprising or consisting of baricitinib, fedratinib, ruxolitinib, and any mixes thereof.

Other agents that may be administered to a subject with a nidovirus infection or a picornavirus infection as described hereinabove, preferably a coronavirus infection, in particular a SARS-CoV-2 infection causing COVID-19, include, without being limited to, BXT-25, brilacidin, dehydroandrographolide succinate, APN01, fingolimod, methylprednisolone, thalidomide, bevacizumab, sildenafil citrate, interferon (such as interferon beta-1a (IFN-β-1a), interferon beta-1b (IFN-β-1b) and peginterferon beta-1a), carrimycin, angiotensin receptor-blocker (ARB), angiotensin-converting-enzyme inhibitors (ACE-I), losartan, bevacizumab, CD24Fc.

In one embodiment, the at least one further pharmaceutically active agent is selected from the group comprising or consisting of BXT-25, brilacidin, dehydroandrographolide succinate, APN01, fingolimod, methylprednisolone, thalidomide, bevacizumab, sildenafil citrate, interferon (such as interferon beta-1a (IFN-β-1a), interferon beta-1b (IFN-β-1b) andpeginterferon beta-1a), carrimycin, angiotensin receptor-blocker (ARB), angiotensin-converting-enzyme inhibitors (ACE-), losartan, bevacizumab, CD24Fc, and any mixes thereof.

Another object of the present invention is a method for treating a nidovirus infection or a picornavirus infection as described hereinabove, preferably a coronavirus infection, in a subject in need thereof, comprising or consisting of administering to the subject a 2-aminoarylthiazole derivative, preferably masitinib, or a pharmaceutically acceptable salt or solvate thereof, as described hereinabove.

In one embodiment, the method of the invention comprises or consists of administering a pharmaceutical composition as described herein, said pharmaceutical composition comprising, consisting essentially of, or consisting of a 2-aminoarylthiazole derivative, preferably masitinib, or a pharmaceutically acceptable salt or solvate thereof, and at least one pharmaceutically acceptable excipient.

In one embodiment, the method of the invention comprises or consists of administering the 2-aminoarylthiazole derivative, preferably masitinib, or a pharmaceutically acceptable salt or solvate thereof, in combination with isoquercetin or quercetin, preferably isoquercetin.

In one embodiment, the method of the invention comprises administering at least one further pharmaceutically active agent as described hereinabove.

In one embodiment, the method of the invention is for treating a SARS-CoV-2 infection causing COVID-19 as described hereinabove. In one embodiment, the method of the invention is for preventing and/or treating COVID-19 associated pneumonia and/or COVID-19 associated acute respiratory distress syndrome (ARDS) in a subject in need thereof as described hereinabove.

Another object of the present invention is a pharmaceutical composition for treating or for use in the treatment of a nidovirus infection or a picornavirus infection as described hereinabove in a subject in need thereof, wherein said pharmaceutical composition comprises, consists essentially of, or consists of a 2-aminoarylthiazole derivative, preferably masitinib, or a pharmaceutically acceptable salt or solvate thereof, and at least one pharmaceutically acceptable excipient. According to one embodiment, the present invention relates to a pharmaceutical composition for treating or for use in the treatment of a coronavirus infection, in particular a SARS-CoV-2 infection causing COVID-19, in a subject in need thereof, wherein said pharmaceutical composition comprises, consists essentially of, or consists of a 2-aminoarylthiazole derivative, preferably masitinib, or a pharmaceutically acceptable salt or solvate thereof, and at least one pharmaceutically acceptable excipient.

Pharmaceutically acceptable excipients, for example, vehicles, adjuvants, carriers or diluents, are well-known to those who are skilled in the art and are readily available to the public. Typically, the pharmaceutically acceptable excipient is one that is chemically inert to the active compound(s) (also referred to as active agent(s) or active ingredient(s)) and one that has no detrimental side effects or toxicity under the conditions of use.

In one embodiment, the pharmaceutical composition comprises, consists essentially of, or consists of masitinib, or a pharmaceutically acceptable salt or solvate thereof, and at least one pharmaceutically acceptable excipient. In one embodiment, the pharmaceutical composition consists of masitinib, or a pharmaceutically acceptable salt or solvate thereof, and at least one pharmaceutically acceptable excipient.

In one embodiment, the pharmaceutical composition of the invention further comprises isoquercetin or quercetin, preferably isoquercetin. Thus, in one embodiment, the pharmaceutical composition comprises, consists essentially of, or consists of masitinib, or a pharmaceutically acceptable salt or solvate thereof; isoquercetin or quercetin, preferably isoquercetin; and at least one pharmaceutically acceptable excipient. In one embodiment, the pharmaceutical composition consists of masitinib, or a pharmaceutically acceptable salt or solvate thereof; isoquercetin or quercetin, preferably isoquercetin; and at least one pharmaceutically acceptable excipient. In one embodiment, the pharmaceutical composition consists of masitinib, or a pharmaceutically acceptable salt or solvate thereof, isoquercetin, and at least one pharmaceutically acceptable excipient.

In one embodiment, the pharmaceutical composition of the invention is for treating or for use in the treatment of a nidovirus infection or a picornavirus infection, preferably a coronavirus infection as described hereinabove, in combination with isoquercetin or quercetin, preferably isoquercetin.

Another object of the present invention is thus a pharmaceutical composition for treating or for use in the treatment of a nidovirus infection or a picornavirus infection as described hereinabove, preferably a coronavirus infection, in a subject in need thereof in combination with isoquercetin or quercetin, preferably isoquercetin, wherein said pharmaceutical composition comprises, consists essentially of, or consists of a 2-aminoarylthiazole derivative, preferably masitinib, or a pharmaceutically acceptable salt or solvate thereof, and at least one pharmaceutically acceptable excipient.

Another object of the present invention is a pharmaceutical composition in combination with isoquercetin or quercetin, preferably isoquercetin, for treating or for use in the treatment of a nidovirus infection or a picornavirus infection as described hereinabove, preferably a coronavirus infection, in a subject in need thereof, wherein said pharmaceutical composition comprises, consists essentially of, or consists of a 2-aminoarylthiazole derivative, preferably masitinib, or a pharmaceutically acceptable salt or solvate thereof, and at least one pharmaceutically acceptable excipient.

In one embodiment, the pharmaceutical composition of the invention is for treating or for use in the treatment of a SARS-CoV-2 infection causing COVID-19 as described hereinabove.

In one embodiment, the pharmaceutical composition of the invention is for preventing and/or treating or for use in the prevention and/or treatment of COVID-19 associated pneumonia and/or COVID-19 associated acute respiratory distress syndrome (ARDS) in a subject in need thereof.

Another object of the present invention is the use of a 2-aminoarylthiazole derivative, preferably masitinib, or a pharmaceutically acceptable salt or solvate thereof, for the manufacture of a medicament for the treatment of a nidovirus infection or a picornavirus infection as described hereinabove, preferably a coronavirus infection, in a subject in need thereof.

In one embodiment, the present invention relates to the use of a 2-aminoarylthiazole derivative, preferably masitinib, or a pharmaceutically acceptable salt or solvate thereof, in combination with isoquercetin or quercetin, for the manufacture of a medicament for the treatment of a nidovirus infection or a picornavirus infection as described hereinabove, preferably a coronavirus infection, in a subject in need thereof.

In one embodiment, the present invention relates to the use of a 2-aminoarylthiazole derivative, or a pharmaceutically acceptable salt or solvate thereof, as described hereinabove for the manufacture of a medicament for the treatment of nidovirus infection or a picornavirus infection as described hereinabove, preferably a coronavirus infection, in a subject in need thereof, wherein said medicament is for administration in combination with isoquercetin or quercetin.

In one embodiment, said medicament is for administration in combination with at least one further pharmaceutically active agent as described hereinabove.

In one embodiment, the coronavirus infection is a SARS-CoV-2 infection causing COVID-19 as described hereinabove. In one embodiment, said medicament is for preventing and/or treating COVID-19 associated pneumonia and/or COVID-19 associated acute respiratory distress syndrome (ARDS) in a subject in need thereof.

### BRIEF DESCRIPTION OF THE DRAWINGS

FIGS. 1A-1C are a combination of graphs illustrating the effect of masitinib, isoquercetin and a combination of masitinib and isoquercetin on non-senescent cells. FIG. 1A shows the dose-dependent effect of masitinib alone (from 0.1 to 2 μM) on the viability of non-senescent cells. FIG. 1B shows the dose-dependent effect of isoquercetin alone (from 1 to 20 μM) on the viability of non-senescent cells. FIG. 1C shows the dose-dependent effect of a combination of masitinib (from 0.1 to 2 μM) and isoquercetin (from 1 to 20 μM) on the viability of non-senescent cells.

FIGS. 2A-2C are a combination of graphs illustrating the effect of masitinib, isoquercetin and a combination of masitinib and isoquercetin on senescent cells. FIG. 2A shows the dose-dependent effect of masitinib alone (from 0.1 to 2 μM) on the viability of senescent cells. FIG. 2B shows the dose-dependent effect of isoquercetin alone (from 1 to 20 μM) on viability of senescent cells. FIG. 2C shows the dose-dependent effect of a combination of masitinib (from 0.1 to 2 μM) and isoquercetin (from 1 to 20 μM) on the viability of senescent cells.

FIG. 3 is a graph showing average percent of OC43 infected cells per well against increasing concentrations of masitinib. Individual measurements are shown as semi-transparent circles (some circles overlap).

FIG. 4 is a line graph showing masitinib inhibition of OC43 replication in primary human airway epithelial cells with an EC50 of 0.58 μM.

FIG. 5 is a graph showing results of masitinib treatment of A549 cells over-expressing ACE2 pre-treated with masitinib at multiple concentrations for 2 hours, infected with SARS-CoV-2 (MOI 0.5) and incubated for 2 days. Cells were stained for the presence of the spike protein and the percent of infected cells was analyzed. Individual measurements are shown as semi-transparent circles (some circles overlap).

FIG. 6 is a graph showing the effect of masitinib on SARS-CoV-2 progeny production. Cells were treated with 10 μM of masitinib for 2 hours, infected with SARS-CoV-2 (MOI=0.5) and cell supernatants were collected for titration 2 days later, n=3. Individual measurements are shown as semi-transparent circles. Masitinib showed a statistically significant (p-values<0.001, one-tailed t-test, FDR-corrected) reduction in viral titers.

FIGS. 7A-E are a set of graphs illustrating the inhibitory activity of masitinib on SARS-CoV-2 main protease known as 3CLpro, M^{pro} or nsp5. FIG. 7A is a bar graph showing the results of a FlipGFP reporter assay performed to assess the inhibition of 3CLpro by masitinib at a single concentration (10 μM). Individual measurements are shown in circles. Bars depict mean±s.e. Masitinib treatment completely inhibited 3CLpro activity. FIG. 7B is a dose-response curve for 3CLpro inhibition by masitinib using the FlipGFP reporter assay, n=6. Individual measurements are shown as circles. FIG. 7C is a dose-response curve for 3CLpro inhibition by masitinib using a luciferase reporter assay, n=3. Individual measurements are shown as circles. FIG. 7D is a dose-response curve for 3CLpro inhibition by masitinib in a cell-free assay using purified 3CLpro and a flurogenic peptidic substrate, n=3. Individual measurements are shown as circles. FIG. 7E is a line graph showing in vitro characterization of masitinib inhibition of 3CL in the presence of different substrate (S) concentrations, as indicated. Masitinib is a competitive inhibitor of 3CL activity with a Ki value of 2.58 μM.

FIGS. 8A-B illustrate the binding of masitinib to SARS-CoV-2 main protease known as 3CLpro, M^{pro} or nsp5. FIG. 8A shows the dimer formation, domain structure, and masitinib binding site of SARS-CoV-2 3CLpro. In monomer A, the inhibitor masitinib is drawn in stick format, bound to the active site between D1 and D2. The sites of three binding pockets S1, S2, and S4 are marked. FIG. 8B presents the interaction of masitinib with 3CLpro. The ribbon diagram shows details of some interactions formed between masitinib and 3CLpro at the active site. Key pocket forming or interacting residues of 3CLpro are also presented in stick format with their C atoms. Hydrogen bonds are drawn in dashed lines. The two catalytic residues are marked by asterisks.

FIGS. 9A-B are a set of graphs illustrating the inhibitory effect of masitinib on picornaviruses. FIG. 9A is a bar graph showing results of a luciferase reporter assay performed to investigate masitinib ability to inhibit the proteolytic activity of picornaviruses 3C (derived from coxsackievirus B3 (CVB3)). n=6, p-value=7×10-6 (one-tailed t-test). FIG. 9B presents bar graphs showing the results after Huh7 cells were treated with 10 M masitinib for 2 hours, infected with coxsackievirus B3 (CVB3) or human rhinoviruses 2, 14 and 16 (HRV2, HRV14, HRV16) at an MOI of 0.01 and the supernatant collected for titration 24 hours later. n=3, p-values<0.001 (one-tailed t-test, FDR-corrected).

FIG. 10 presents bar graphs showing that masitinib (10 μM) did not show a significant effect on cells infected by influenza A virus (IAV, Orthomyxoviridae), measles virus (MeV, Paramyxoviridae), lymphocytic choriomeningitis virus (LCMV) and Chikungunya virus (CHIKV, Togaviridae). n=3 for all except LCMV (n=2). p-values>0.07 (one-tailed t-test, FDRcorrected).

FIGS. 11A-B present dot plots showing SARS-CoV-2 viral loads in mice lungs (11A) and in mice nasal turbinates (11B), 4 and 6 days post infection. Mice were treated with masitinib (25 or 50 mg/kg, bid, ip) or PBS.

FIG. 12 is a line graph showing clinical score of mice, 1-6 days post infection. Mice were treated with masitinib (25 or 50 mg/kg, bid, ip) or PBS.

### EXAMPLES

The present invention is further illustrated by the following examples.

### Example 1: In Vitro Senolytic Effect of Masitinib and Isoquercetin

Materials and Methods

Material

BV2 cells are retroviral-immortalized microglia-like cells, which are used as a model for cellular senescence. BV2 cells were cultured in Dulbecco's modified Eagle's medium supplemented with 10% heat-inactivated fetal bovine serum (hiFBS) and plated in 6-well multiwell plates for treatment and flow cytometry analysis.

Masitinib (masitinib mesilate) was obtained from AB Science (Paris, France) and prepared as solutions of 0.1-2 μM in DMSO.

Isoquercetin was prepared as solutions of 1-20 μM in DMSO.

Methods

Senescence Model

BV2 cells were treated with temozolomide (TMZ), an alkylating agent inducing DNA damage, in order to induce senescence of the cells. For senescence induction studies, cells were plated and treated twice with increasing doses (10-150 μM) of TMZ during 5 hours every 24 hours. After exposure of BV2 cells to two successive treatments with TMZ, their proliferation was reduced and the cells developed a characteristic senescent phenotype with enlarged size and flat-granulated shape. The senescent phenotype of BV2 cells after TMZ-induced genotoxicity was further confirmed by measuring the number of cells displaying β-gal activity, a well-recognized marker of cellular senescence. After the second TMZ-treatment, flow cytometry analysis was thus carried out using a 5-Dodecanoylaminofluorescein Di-β-D-Galactopyranoside (C12FDG) kit to measure β-galactosidase activity by the alkalinization of lysosomes as described by the manufacturer's instructions (Thermo Fisher Scientific, #D2893). TMZ induced a dose-dependent increase in the number β-gal+ cells. Up to 60% of β-gal+BV2 cells, i.e., senescent cells, were obtained at a TMZ concentration of 100 μM (data not shown).

Treatment with Masitinib, Isoquercetin, or a Combination of Masitinib and Isoquercetin

For cellular viability analysis of non-senescent cells (BV2 cells) and senescent cells (BV2 cells pretreated with TMZ), cells were plated in 96-well multiwell plates during 72 hours. Cells were treated with increasing doses of isoquercetin (1-20 μM in DMSO), masitinib (0.1-2 μM in DMSO) or with the combination of both isoquercetin and masitinib to study any potential synergistic effects. After 48 hours of treatment, cell viability was analyzed by sulforhodamine B (SRB) assay. The optical density (OD) of each well was read in a 96-well plate reader at 540 nm. The OD of the SRB solution is directly proportional to the cell number.

Results

BV2 cells treated with TMZ, a model well-suited for the screening of senolytic drugs, were used for assessing the effect of masitinib alone, isoquercetin alone, or a combination of masitinib and isoquercetin, in reducing the viability of senescent cells.

BV2 cells pre-treated with TMZ were thus incubated with masitinib alone, isoquercetin alone, or with a combination of masitinib and isoquercetin. As a control, non-senescent proliferating BV2 cells were incubated in the same conditions with masitinib alone, isoquercetin alone, or with a combination of masitinib and isoquercetin.

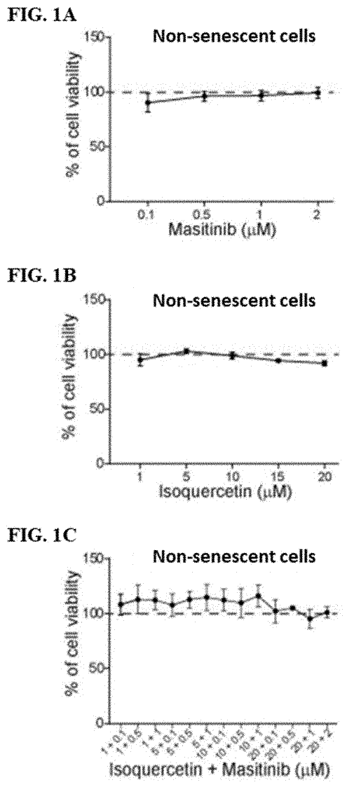

As shown in FIG. 1, when non-senescent proliferating BV2 cells were exposed 48 h to masitinib (FIG. 1A), to isoquercetin (FIG. 1B) or to a combination of masitinib and isoquercetin (FIG. 1C), there was no significant change in cell viability, even at high concentrations of masitinib and/or isoquercetin, as estimated by flow cytometry analysis of viable cells stained with sulforhodamine B (SRB).

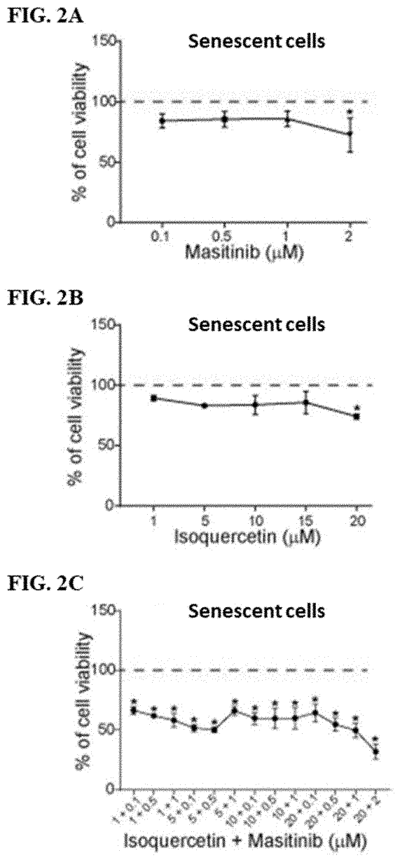

As shown in FIG. 2, exposure of senescent BV2 cells to either masitinib alone (FIG. 2A) or isoquercetin alone (FIG. 2B) only induced a modest reduction in cell viability, except at high concentrations of masitinib (2 μM) or isoquercetin (20 μM). By contrast, the number of viable TMZ-induced senescent BV2 cells was significantly reduced when senescent BV2 cells were exposed 48 h to a combination of masitinib and isoquercetin (FIG. 2C). The senolytic effect was noticeable even at low concentrations of both masitinib and isoquercetin (0.1 μM masitinib+1 μM of isoquercetin) with a reduction of about 30% in senescent cell viability. Strikingly, the senolytic effect of the combination of masitinib and isoquercetin was significantly greater than the added senolytic effects of masitinib alone and isoquercetin alone. For example, the combination of 2 μM masitinib and 20 μM isoquercetin induced a reduction of about 70% in senescent cell viability (FIG. 2C), while 2 μM masitinib alone and 20 μM isoquercetin alone each induced a reduction of about 25% in senescent cell viability (FIG. 2A-B).

In conclusion, the data obtained with BV2 cells show that masitinib and isoquercetin can each selectively and significantly induce a loss of viability in senescent cells at higher concentrations (e.g., 2 μM of masitinib and 20 μM of isoquercetin). The data obtained with BV2 cells also show that the combination of masitinib and isoquercetin selectively and significantly induce a loss of viability in senescent cells, even at low concentrations (e.g., 0.1 μM of masitinib and 1 μM of isoquercetin). Moreover, the data obtained with BV2 cells show that the senolytic effect of the combination of masitinib and isoquercetin is synergistic, that is to say masitinib and isoquercetin act in synergy in selectively and significantly inducing a loss of viability in senescent cells.

### Example 2: Clinical Trial Investigating the Efficacy of a Combination of Masitinib and Isoquercetin for the Treatment of COVID-19

A randomized, double-blind, placebo-controlled clinical trial is described herein, aiming at assessing the safety and efficacy of combinations of masitinib and isoquercetin (with best supportive care) for the treatment of COVID-19 in hospitalized patients.

The overall objective of the study in adult patients hospitalized with COVID-19 is to evaluate the efficacy of a combination of masitinib and isoquercetin.

The study is a randomized, double-blind, placebo-controlled clinical trial with two distinct patient groups defined according to severity of disease (as defined by the World Health Organization (WHO) criteria of severity of COVID-19), which can also broadly be categorized by clinical management of disease, namely, no requirement of admission to intensive care unit (ICU) (Group 1) versus requirement of admission to ICU (Group 2). Each patient group will have a separate control arm, therefore bringing the total to 4 treatment-arms.

Overall, 120 patients are to be recruited; with a set of 60 patients per group, wherein 30 patients are randomized in a 1:1 ratio to either the masitinib/isoquercetin (i.e., combination of masitinib and isoquercetin, with best supportive care) arm or the control arm (placebo masitinib and placebo isoquercetin, with best supportive care).

Group 1: Patients not Requiring ICU Admission (at the Time of Randomization)
30 patients will be randomized to receive masitinib/isoquercetin with best supportive care (excluding hydroxychloroquine and chloroquine). The oral dose of masitinib is 3 mg/kg/day (mg per kilo body weight per day) or 4.5 mg/kg/day. If safety as assessed by the Data Safety Monitoring Board (DSMB) is acceptable, patients may receive masitinib 3 mg/kg/day for at least 4 days, preferably for at least 2 days, then 4.5 mg/kg/day.
At least 30 patients with matching baseline characteristics will be included in the control arm and will receive placebo masitinib and placebo isoquercetin with best supportive care (excluding hydroxychloroquine and chloroquine).

Best Supportive Care is best available therapy at the choice of the investigator including, but not limited to, oxygenation, analgesics, anti-thrombotics, anti-viral drugs and biologics drugs.

Group 2: Patients Requiring ICU Admission
30 patients will be randomized to receive masitinib/isoquercetin with best supportive care (excluding hydroxychloroquine and chloroquine). The oral dose of masitinib will be 3 mg/kg/day or 4.5 mg/kg/day. If safety as assessed by the DSMB is acceptable, patients may receive masitinib 3 mg/kg/day for at least 4 days, preferably for at least 2 days, then 4.5 mg/kg/day. The patients may receive or not steroids depending on the local procedures.
At least 30 patients with matching baseline characteristics will be included in the control arm and will receive placebo masitinib and placebo isoquercetin with best supportive care (excluding hydroxychloroquine and chloroquine).

The recommended oral dose for the combination masitinib/isoquercetin is:
Masitinib: patients receive a daily masitinib dose of 3 mg/kg/day (mg per kilo body weight per day) or 4.5 mg/kg/day. If safety as assessed by the DSMB is acceptable, patients may receive a daily masitinib dose of 3 mg/kg/day for at least 4 days, preferably for at least 2 days, then a daily masitinib dose of 4.5 mg/kg/day thereafter.
Isoquercetin: daily isoquercetin dose of 1 g/day by oral route.

Masitinib/isoquercetin is to be taken until 24 h after cessation of oxygen therapy or hospital discharge, preferably with a minimum of 7 days of treatment.

The duration of the study is 90 days.

The WHO criteria of severity of COVID-19 are as follows:
mild: cases showing mild clinical symptoms, with no sign of pneumonia on imaging;
moderate: cases showing fever and respiratory symptoms with radiological findings of pneumonia; and requiring oxygen (O₂): 3 L/min<O₂<5 L/min;
severe: cases meeting any of the following criteria:
   respiratory distress (respiratory rate (RR)≥30 breaths/min);
   oxygen saturation (SpO₂)≤93% at rest in ambient air; or SpO₂≤97% with O₂>5 L/min;
   ratio of artery partial pressure of oxygen/inspired oxygen fraction (PaO₂/FiO₂)≤300 mmHg (1 mmHg=0.133 kPa), PaO₂/FiO₂ in high-altitude areas (at an altitude of over 1,000 meters above the sea level) shall be corrected by the following formula: PaO₂/FiO₂ [multiplied by] [Atmospheric pressure (mmHg)/760]; and/or
   chest imaging that showed obvious lesion progression within 24-48 hours>50%;
critical: cases meeting any of the following criteria:
   respiratory failure and requiring mechanical ventilation;
   shock; and/or
   other organ failure that requires ICU care.

Inclusion Criteria:
1. Laboratory-confirmed SARS-CoV-2 infection as determined by polymerase chain reaction (PCR), or other commercial or public health assay in any specimen ≤72 hours and/or CT scan prior to randomization (following typical radiological findings (ground glass abnormalities, and absence of lymphadenopathy, pleural effusion, pulmonary nodules, lung cavitation))
2. Hospitalized patients for the treatment of COVID pneumopathy
3. Male or female adult ≥18 years of age at time of enrolment
4. Patients belonging to one of the two following groups:
   Group 1: patients not requiring ICU at initial hospital admission with moderate and severe pneumopathy according to the WHO criteria of severity of COVID-19:
      Moderate cases (score of 4 on the modified WHO 7-point progression scale as described in Table 2 hereinabove)

Cases meeting all of the following criteria:
showing fever and respiratory symptoms with radiological findings of pneumonia; and
requiring between 3 L/min and 5 L/min of oxygen to maintain SpO₂>97%. Or

Cases of moderate pneumopathy defined by all of the following criteria:
requiring more than 3 L/min of oxygen;
score on the WHO 10-point progression scale=5; and
no non-invasive ventilation (NIV) or high flow oxygen.
Severe cases (score of 5 on the modified WHO 7-point progression scale as described in Table 2 hereinabove)

Cases meeting any of the following criteria:
respiratory distress (RR≥30 breaths/min);
SpO₂≤93% at rest in ambient air; or SpO₂≤97% with O₂>5 L/min; and/or
PaO₂/FiO₂ 300 mmHg.
Group 2: patients requiring ICU based on criteria of severity of COVID pneumopathy:
   respiratory failure and requiring mechanical ventilation; and
   no do-not-resuscitate order (DNR order).
5. Patient with body weight >45 kg and BMI≥18 and ≤35 kg/m2.

The primary endpoint and secondary endpoints will depend on the group of patients tested.

For Group 1 patients (not requiring ICU):
Co Primary Endpoints
1. Survival without need of ventilator utilization, including non-invasive ventilation (NIV), at day 14. Thus, events considered are the requirement of ventilator utilization (including NIV), or death. New DNR order will be considered as an event at the date of the DNR.
2. Early end point: score on WHO 10-point progression scale ≤5 (as described in Table 1 hereinabove) at day 4 or clinical status of patients at day 15 using the modified WHO 7-point progression scale (as described in Table 2 hereinabove).
Secondary end-points for Group 1 will be the following:
Score on WHO 10-point progression scale at 4, 7 and 14 days;
Overall survival at 14, 28 and 90 days;
Time to transfer to ICU;
Time to ventilator utilization or NIV or high flow;
Time to discharge;
Time to oxygen supply independency;
Time to negative viral excretion;
Biological parameters improvement: estimated glomerular filtration rate (eGFR), C-reactive protein (CRP), myoglobin, creatine phosphokinase (CPK), cardiac troponin, ferritin, lactate, cell blood count, liver enzymes, lactate dehydrogenase (LDH), D-Dimer, albumin, fibrinogen, triglycerides, coagulation tests, urine electrolyte, creatinuria, proteinuria, uricemia, IL6, procalcitonin, immunophenotype, and exploratory tests;
Clinical status using the following ordinal scale corresponding to the modified WHO 7-point progression scale as described in Table 2 hereinabove: 1. not hospitalized, no limitations on activities; 2. not hospitalized, limitation on activities; 3. hospitalized, not requiring supplemental oxygen; 4. hospitalized, requiring supplemental oxygen; 5. hospitalized, on non-invasive ventilation or high flow oxygen devices; 6. hospitalized, on invasive mechanical ventilation or ECMO; 7. Death.

For Group 2 patients (requiring ICU):
Co Primary Endpoints
1. Cumulative incidence of successful tracheal extubation (defined as duration extubation >48 h) at day 14. Death or DNR order will be considered as a competing event.
2. Early end point: score on WHO 10-point progression scale ≤7 (as described in Table 1 hereinabove) at day 4.
Secondary end-points for Group 2 will be the following:
   Score on WHO 10-point progression scale at 4, 7 and 14 days;
   Overall survival at 14, 28 and 90 days;
   28-day ventilator-free days;
   Evolution of PaO₂/FiO₂ ratio;
   Respiratory acidosis at day 4 (arterial blood pH of ≤7.25 with a partial pressure of arterial carbon dioxide [Paco₂] of ≥60 mm Hg for >6 hours);
   Time to oxygen supply independency;
   Duration of hospitalization;
   Time to negative viral excretion;
   Time to ICU discharge;
   Time to hospital discharge;
   Biological parameters improvement (eGFR, CRP, cardiac troponin, urine electrolyte and creatinine, proteinuria, uricemia, IL6, myoglobin, kidney injury Molecule-1 (KIM-1), neutrophil gelatinase-associated lipocalin (NGAL), CPK, ferritin, lactate, cell blood count, liver enzymes, LDH, D-Dimer, albumin, fibrinogen, triglycerides, coagulation tests (including activated partial thromboplastin time), procalcitonin;
   Clinical status using the following ordinal scale corresponding to the modified WHO 7-point progression scale as described in Table 2 hereinabove: 1. not hospitalized, no limitations on activities; 2. not hospitalized, limitation on activities; 3. hospitalized, not requiring supplemental oxygen; 4. hospitalized, requiring supplemental oxygen; 5. hospitalized, on non-invasive ventilation or high flow oxygen devices; 6. hospitalized, on invasive mechanical ventilation or ECMO; 7. Death;
   Rate of renal replacement therapy;
   Ventilation parameters.

Criteria for safety assessment include:
Number of serious adverse events
Cumulative incidence of serious adverse events
Cumulative incidence of Grade 3 and 4 adverse events
Investigational medication discontinuation (for any reason)

### Example 3: Clinical Trial Investigating the Efficacy of Masitinib as a Single Agent for the Treatment of COVID-19

A randomized, double-blind, placebo-controlled, phase 2 clinical trial is described herein, aiming at evaluating the anti-viral efficacy of masitinib in patients with symptomatic mild to moderate COVID-19.

The overall objective of the study is to evaluate the anti-viral efficacy of 3 different dosages of masitinib in patients with symptomatic mild to moderate COVID-19.

All patients are to receive best supportive care in addition, patients will be randomized into one of the following 3 arms
Masitinib 3.0 mg/kg/day for 10 days versus corresponding placebo,
Masitinib 3.0 mg/kg/day for 2 days then 4.5 mg/kg/day for 8 days versus corresponding placebo,
Masitinib 3.0 mg/kg/day for 2 days then 4.5 mg/kg/day for 2 days then 6.0 mg/kg/day for 6 days versus corresponding placebo.

For each arm, hydroxychloroquine, chloroquine and remdesivir are excluded. Best supportive care is best available therapy at the choice of the investigator including, but not limited to, antipyretic, corticosteroids, oxygenation, analgesics, anti-thrombotics and approved anti-viral drugs for COVID-19. Treatments will be administered for 10 days. Patients will be followed for 1 month.

78 patients are to be recruited. For each arm, 20 patients are to be treated with masitinib and 6 patients with placebo. 50% of patients are to be included in active and control arm with score 2 or 3 on the 10-score WHO clinical progression scale (as described in Table 1 hereinabove) and 50% of patients are to be included in active and control arm with score 4 or 5 on the 10-score WHO clinical progression scale (as described in Table 1 hereinabove).

Inclusion Criteria:
1. Male or non-pregnant female with symptomatic ambulatory mild COVID-19 (score 2 and 3 on the 10-score WHO clinical progression scale as described in Table 1 hereinabove) either with
   Age ≥75 years
   OR 65 years <age <74 years with the following comorbidities:
      Complicated arterial hypertension
      Class I obesity: BMI of 30 to ≤35 kg/m2
      Diabetes
      Obstructive lung disease or respiratory failure
OR Hospitalized male or non-pregnant female adult ≥18 years of age at time of enrolment with COVID-19 with score 4 on the 10-score WHO clinical progression scale as described in Table 1 hereinabove with the following comorbidities:
   Complicated arterial hypertension
   Class I obesity: BMI of 30 to ≤35 kg/m2
   Diabetes
   Obstructive lung disease or respiratory failure
OR Hospitalized male or non-pregnant female adult ≥18 years of age at time of enrolment with COVID-19 with score 5 on the 10-score WHO clinical progression scale as described in Table 1 hereinabove.
2. Has symptoms consistent with COVID-19, as determined by investigator, with onset ≤5 days before randomization.
3. Has positive test for COVID-19 (by validated SARS-CoV-2 RT-PCR, or other molecular diagnostic assay, using an appropriate sample such as nasal, oropharyngeal, or saliva)≤72 hours prior to randomization and no alternative explanation for current clinical condition.
4. Patient with body weight >45 kg and BMI ≥18 and ≤35 kg/m².

The primary objective is to evaluate the efficacy of masitinib in mild and moderate COVID-19 patients based on the viral load of patients after 10-day treatment. The primary endpoint thus is: viral load change at day 4, day 7, and day 10 measured by RT-qPCR in nasal swab.

Criteria for safety assessment include:
Cumulative incidence of serious adverse events (SAEs)
Cumulative incidence of Grade 3 and 4 adverse events (AEs).
Investigational medicinal product discontinuation (for any reason)

### Example 4: In Vitro Antiviral Effect of Masitinib

This example demonstrates that masitinib inhibits nidoviruses and picornaviruses.

Materials and Methods

Material

Cells

A549 expressing H2B-mRuby were generated by first infecting A549 cells (ATCC CCL-185) with a lentivirus (carrying H2B-mRuby), and FACS-sorting mRuby+ cells. They were maintained as a polyclonal population and grown in DMEM+10% BCS (bovine calf serum). These cells were used for all OC43 infections (i.e., infections with HCoV-OC43). Ace2-A549 cells (Blanco-Melo et al., Cell, 181: 1036-1045.e9 (2020), incorporated by reference herein) were used for SARS-CoV-2 infections. They were maintained in DMEM+10% FBS (fetal bovine serum). African green monkey kidney cells (Vero E6) were maintained in DMEM supplemented with 10% FBS, 1% penicillin-streptomycin and 1% HEPES. Huh7 cells were used for picornaviruses infections (i.e., infections with coxsackievirus B3 (CVB3) or one of human rhinoviruses 2, 14 and 16 (HRV 2, 14, 16)). MDCK-SIAT1-TMPRSS2 cells were used for influenza A virus (IAV) infections. A549 cells maintained in 50:50 DMEM:F-12 media supplemented with 10% FBS and 1% penicillin-streptomycin were used for lymphocytic choriomeningitis virus (LCMV) infections.

Viruses

OC43 (i.e., HCoV-OC43) was obtained from ATCC (VR-1558) grown and titrated on A549-mRuby cells. SARS-CoV-2 (nCoV/Washington/1/2020) was provided by the National Biocontainment Laboratory, Galveston, Tex. VeroE6 cells were used to propagate and titer SARS-CoV-2. Coxsackievirus B3 or CVB3 (Nancy strain), human rhinoviruses (HRV) 2, 14, and 16 were derived from full-length infectious clones and generated in Vero cells (NR-10385, BEI Resources, NIAID, NIH). Recombinant lymphocytic choriomeningitis virus (rLCMV) based on the Armstrong 53b strain was generated as previously described (Flatz et al., Proc. Natl. Acad. Sci. U.S.A., 103: 4663-4668 (2006) and Ziegler et al., PLoS Pathog., 12: e1005501 (2016), each of which is incorporated by reference herein). Working stocks were generated in Vero E6 cells, and the same cells were used to measure virus titers. The measles virus (MeV) used was derived from the molecular cDNA clone of the Moraten/Schwartz vaccine strain (del Valle et al., J. Virol., 81: 10597-10605 (2007), incorporated by reference herein). The recombinant measles virus was engineered to express firefly luciferase as previously described (Munoz-Alfa et al., Viruses, 11: (2019), incorporated by reference herein).

Methods

Drug Screening

A549-mRuby cells were seeded (3,000 cells per well) in nine 384-well plates using Multidrop combi. Cells were seeded in a final volume of 30 L with DMEM+10% BCS. The following day, 20 L of OC43 were added (multiplicity of infection (MOI) 0.3) and incubated at 33° C., 5% CO₂ for 1 hour. 50 nL from the Selleck FDA-approved drug library (cat #L1300, Selleck) were added (1:1,000 dilution). Two columns (32 wells) were left uninfected and two columns were treated with DMSO and virus (no-drug control). Cells were imaged using the IncuCyte S3 to measure cell numbers at day 0. Cells were incubated for 4 days at 33° C., 5% CO₂ and were stained for OC43 nucleoprotein. All the following steps were performed at room temperature. Cells were fixed in 50 L 4% PFA/PBS for 15 min, blocked with 50 l 10% BSA+0.5% Triton X-100 in PBS for 30 minutes, stained with 50 l anti-OC43 (cat #MAB9013, Millipore) diluted 1:2,000 in 2% BSA+0.1% Triton X-100 in PBS for 1 hour, washed with 50 L PBS three times, stained with anti-mouse-AlexaFluor488 diluted 1:1,000 in 2% BSA+0.1% Triton X-100 in PBS for 1 hour, washed with 50 L PBS three times and imaged on the IncuCyte S3 (day 4). The screen was performed twice.

The following parameters were extracted from the images: number of cells at day 0, number of cells at day 4 and total OC43 staining intensity at day 4. For analysis, OC43 staining intensity was normalized to the number of cells in the well and further normalized to the mean of the no-drug controls, which was set to 100. Removed from analysis were compounds that showed significant effect on cell growth. For each plate, a drug was considered as a putative hit if it reduced OC43 staining by over 3 standard deviations from the mean of the no-drug controls. Drugs were considered hits if they were not toxic and reduced OC43 staining by over 3 standard deviations in both repeats. Masitinib was thus identified as a hit, with a mean % staining of OC43 of 12.2 (corresponding to a % staining of OC43 of 14.4 in screen repeat #1 (normalized to no-drug controls in the same plate) and a % staining of OC43 of 10.1 in screen repeat #2 (normalized to no-drug controls in the same plate), and with number of cells at day 4 divided by number of cells at day 0 of 4.3 (repeat #1) and 4.7 (repeat #2).

Dose-Response Analysis for OC43 and SARS-CoV-2 Infection

Dose-response analysis of OC43 infection was done similarly to the drug screening, except cells were seeded at a concentration of 5,000 cells per well and the media contained 2% BCS instead of 10% BCS. OC43 staining was performed 2 days after infection and analyzed similarly to what was described for the drug screening. A sigmoid fit was used to extract EC50 values using Matlab.

All SARS-CoV-2 infections were performed in biosafety level 3 conditions at the Howard T. Ricketts Regional Biocontainment Laboratory. Ace2-A549 cells in DMEM+2% FBS were treated with drugs for 2 hours with 2-fold dilutions beginning at 10 M in triplicate for each assay. Cells were infected with an MOI of 0.5 in media containing the appropriate concentration of drugs. After 48 hours, the cells were fixed using 3.7% formalin, blocked and probed with mouse anti-Spike antibody (GTX632604, GeneTex) diluted 1:1,000 for 4 hours, rinsed and probed with anti-mouse-HRP for 1 hour, washed, then developed with DAB (3,3′diaminobenzidine) substrate 10 minutes. Spike positive cells (n>40) were quantified by light microscopy as blinded samples.

For SARS-CoV-2 plaque titers, cell supernatants from the infection described above were serially diluted (10-fold steps were used) and used to infect Vero E6 cells for 1 hour. Inoculum was removed and 1.25% methylcellulose DMEM solution was added to the cells and incubated for 3 days. Plates were fixed in 1:10 formalin for 1 hour, stained with crystal violet for 1 hour and were counted to determine plaque forming units (PFU)/ml.

FlipGFP SARS-CoV-2 3CLpro Assay

293T cells were seeded 24 hours before transfection on poly-lysine treated plates. The next day, SARS-CoV-2 3CLpro plasmid, FlipGFP coronavirus reporter plasmid, Opti-MEM, and TransIT-LT (Mirus) were combined and incubated at room temperature for 20 minutes before being added to the cells. Masitinib was applied to the cells at the indicated concentrations at the time of transfection. 24 hours after transfection, cells were fixed with 2% PFA at room temperature for 20 minutes and were incubated in 1:10,000 Hoescht 33342 (Life Technologies) in PBS at 4° C. overnight. Quantification was performed by using the CellInsight CX5 (Thermo Scientific) equipment.

3CLpro Luciferase Reporter Assay

Approximately 16 hours before transfection, 293T cells were plated in 96-well plates and grown to 70-80% confluency overnight. The next day, the cells were transfected with 37.5 ng pGlo-30F-VRLQS, 37.5 ng SARS-CoV2 3CLpro, and 2.5 ng pRL-TK (Promega) using Lipofectamine 2000 (Invitrogen) using manufacture's recommendations. After 18 hours, masitinib (0-10 M) was added to the cells and incubated for an additional 6 hours before luciferase readout on a Biotek Synergy plate reader as previously described (Kilianski et al., J. Virol., 87: 11955-11962 (2013), incorporated by reference herein). Briefly, 40 μL growth media was removed from every well and then 40 μL firefly assay buffer (Triton Lysis Buffer (50 mM Tris, pH 7.0, 75 mM NaCl, 3 mM MgCl2, 0.25% Triton X-100) containing 5 mM DTT, 0.2 mM coenzyme A, 0.15 mM ATP, and 1.4 mg/mL D-luciferin) was added to lyse the cells, and to provide the substrate for firefly luciferase. Firefly luminescence was read 10 minutes later and 40 L Renilla assay buffer (45 mM EDTA, 30 mM sodium pyrophosphate, 1.4 M NaCl, 0.02 mM PTC124, 0.003 mM coelentrazine h (CTZ-h)) was added to stop firefly luciferase activity and provide the substrate for Renilla luciferase. Renilla luminescence was read 2-3 minutes after addition of the buffer. Firefly luciferase luminescence was normalized to the corresponding Renilla luciferase luminescence to generate normalized luminescence.

3CLpro Kinetic Assay

The cell-free inhibition assay was done in triplicates at 25° C. using 96-well plates. Reactions containing the different concentrations of masitinib (0-100 M) and 3CLpro enzyme (125 nM) in Tris-HCl pH 7.3, 1 mM EDTA, 2 mM DTT were incubated for 20 minutes. Reactions were then initiated with 5-FAM-TSATLQSGFRK(QXL520)-NH2 probe substrate (1.5 μM). Fluorescence emission intensity (excitation: 490 nm; emission: 520 nm) was measured. Data were fit using a sigmoid curve fit in Matlab.

Cloning of 3CLpro (3CL protease) from SARS CoV-2 was based upon the original cloning of SARS-CoV 3CLpro (Xue et al., J. Mol. Biol., 366: 965-975 (2007), incorporated by reference herein). The gene coding for 3CLpro from SARS CoV-2 was cloned between an upstream MBP and a downstream sequence of GPHHHHHH. Detailed cloning of pCSGID-Mpro carrying 3CLpro from SARS CoV-2 is described in Kneller et al. (Kneller et al., Nat. Commun., 11: 3202 (2020), incorporated by reference herein).

pCSGID-Mpro was transformed into 100 mL of E. coli BL21(DE3)-Gold (Strategene) under selection of ampicillin (150 mg/L) and grown overnight at 37° C. The starter was then transferred to 4 L of LB-Miller culture and was grown at 37° C. with constant shaking (190 rpm). After reaching an OD600 of ˜1, the shaker was set to 4° C. When temperature reached 18° C., IPTG and K₂HPO₄ was added to 0.2 mM and 40 mM respectively and the culture was marinated at 18° C. The cells were spun down at 4000 g, resuspended in lysis buffer (500 mM NaCl, 5% (v/v) glycerol, 50 mM HEPES pH 8.0, 20 mM imidazole pH 8.0, 1 mM TCEP) and kept frozen at −80° C.

Bacterial cells were lysed by sonication and debris were removed by centrifugation at 25,400×g for 60 min at 4° C. The clarified supernatant was mixed with 3 mL of Ni²⁺ Sepharose (GE Healthcare Life Sciences) equilibrated with lysis buffer. The suspension was applied to a Flex-Column (420400-2510) which was connected to a Vac-Man vacuum manifold. Unbound protein was washed out using controlled suction lysis buffer (160 ml). 3CLpro was eluted using 15 mL of buffer containing 500 mM NaCl, 5% (v/v) glycerol, 50 mM HEPES pH 8.0, 500 mM imidazole pH 8.0 and 1 mM TCEP. The fractions containing 3CLpro were pooled, and rhinovirus 3C His6 tagged protease was added at a 1:25 protease:protein ratio and incubated at 4° C. overnight to cleave the C-terminal His6 tag, resulting in a 3CLpro with an authentic N and Ctermini. 10 kDa MWCO filter (Amicon-Millipore) was used to concentrate the protein solution, which was subsequently applied to Superdex 75 column, pre-equilibrated with lysis buffer. The fractions containing 3CLpro were pooled together and run through 2 mL of Ni resin. The flow through was collected and the lysis buffer was replaced with crystallization buffer (20 mM HEPES pH 7.5, 150 mM NaCl, 2 mM DTT (1,4-Dithiothreitol, Roche, Basel, Switzerland)) using a 10 kDa MWCO filter. 3CLpro solution was concentrated to 49 mg/mL, was aliquoted, frozen and stored at −80° C.

Crystallization of Masitinib with SARS-CoV-2 3CLpro

Crystallizations were carried out using previous protocols (Kim et al., Methods 55, 12-28 (2011), incorporated by reference herein). 3CLpro was mixed with 0.2 M masitinib solution in DMSO. The final protein concentration was 6.25 mg/mL and inhibitor concentration was 8 times higher. This mixture was incubated for 1 hour (at room temperature) and spun down at 12,000×g to remove precipitation. For crystallization, the sitting-drop vapor-diffusion method was utilized via a Mosquito liquid dispenser (TTP LabTech, Royston, UK) in 96-well CrystalQuick plates (Greiner Bio-One, Monroe, N.C., USA) using a protein-to-matrix ratio of 1:1. ProPlex, PACT premier (Molecular dimensions, Cambridge, UK), and TOP96 (Anatrace, Maumee, Ohio, USA) screens were used for crystallization at 16° C. The first thin-plate crystals (obtained one day later in several conditions) were applied as seeding. The best crystals appeared in PACT B7 (0.2 M sodium chloride, 0.1 MES pH 6.0, 20% PEG 6000), TOP96 H8 (0.1 M ammonium acetate, 0.1 M Bis-Tris pH 5.5, 17% PEG 10000) and Top96 F11 (0.1 M Bis-Tris pH6.5; 25% PEG 3350). Crystals selected for data collection were treated in their crystallization buffers supplemented with 10-18% glycerol and were subsequently flash-cooled in liquid nitrogen.

X-Ray Data Collection and Structure Determination

Cryo-cooled crystals (100 K) were measured using single-wavelength X-ray diffraction experiments at the 19-ID beamline of the Structural Biology Center, Advanced Photon Source at Argonne National Laboratory (using the SBCcollect program). Intensities of each data set were integrated, scaled and merged (HKL-3000 program suite was used (Minor et al., Acta Crystallogr. D Biol. Crystallogr., 62: 859-866 (2006), incorporated by reference herein)). The structure of 3CLpro in complex with masitinib was determined using the molecular replacement method (Vagin et al., Acta Crystallogr. D Biol. Crystallogr. 66, 22-25 (2010), incorporated by reference herein)) with an apo form of 3CLpro (PDB code: 7JFQ) as a search template. In the difference Fourier maps, extra electron densities were observed in the substrate binding site of 3CLpro and were subsequently identified as the contribution of masitinib. One data set with a resolution limit up to 1.60 Å was selected for further model rebuilding, including building masitinib into extra densities using the program Coot (Emsley et al., Acta Crystallogr. D Biol. Crystallogr., 60: 2126-2132 (2004), incorporated by reference herein) and refinement using the program phenix.refine (Terwilliger et al., Acta Crystallogr. D Biol. Crystallogr., 68: 861-870 (2012), incorporated by reference herein) (see Table 3 below). MOLPROBITY (Chen et al., Acta Crystallogr. D Biol. Crystallogr., 66: 12-21 (2010), incorporated by reference herein) was used to validate the stereochemistry of the structure (see Table 3 below).

The X-ray structure of 3CLpro-bound masitinib has been deposited to PDF under accession number 7JU7.

TABLE 3
crystallization of masitinib with SARS-CoV-2
3CL protease (also known as M^{pro})

| Summary of crystallographic data | |
|---|---|
| Data collection statistics | M^{pro} + masitinib |
| Space group | C2 |
| Unit cell (Å, °) | a = 98.55, b = 81.23, |
| | c = 51.85, β =114.6 |
| MW Da (residue) | 33,796 (306) |
| Mol (AU) | 1 |
| Wavelength (Å) | 0.9791 |
| Resolution | 1.60 |
| Number of unique reflections | 48,121 |
| Rmerge (%) | 8.4 (87.7) ¹ |
| Completeness (%) | 98.5 (95.4) ¹ |
| Redundancy | 3.5 (2.3) ¹ |
| I/(σ) | 26.3 (1.1) ¹ |
| Solvent content (%) | 56.0 |
| Phasing | |
| Resolution range (Å) | 43.5-3.00 |
| Correlation coefficient² (%) | 0.58 |
| Refinement | |
| Resolution range (Å) | 43.5-1.60 |
| Number of reflections | 47,567 |
| Completeness (%) | 97.1 |
| Rwork/Rfree (%) | 16.8/19.2 |
| No. of Atoms (Protein/HETATM) | 2,363/249 |
| Bond lengths (Å) | 0.016 |
| Bond angles (deg) | 1.431 |
| B-factors (Å²) (main/side chain) | 32.73/52.9 |
| Wilson B-factor (Å²) | 23.98 |
| Molprobity validation | |
| Ramachandran outliners (%) | 0.33 |
| Ramachandran Favored (%) | 98.34 |
| Rotamer outliners (%) | 0.77 |
| Clashscore | 2.72 |
| MolProbity score | 1.06 |
| PDBID | 7JU7 |

¹ Last resolution bin (1.60-1.63 Å);
²Molecular replacement method.

Picornaviruses Infection

Prior to infection, Huh7 cells were pretreated for two hours. Virus was diluted using serum-free DMEM (SFM) to achieve an MOI of 0.01. Cell supernatants (collected at 24 hours post infection) were dilutions in SFM and used to inoculate Vero cells for 10-15 min at 37 C. Cells were incubated for 2 days at 37 C after overlaying them DMEM containing 2% NBCS and 0.8% agarose. Cells were then fixed with 4% formalin and revealed with crystal violet solution (10% crystal violet; Sigma-Aldrich). The number of plaque forming units (PFU/per milliliter) were then calculated.

3C Protease Activity Assay

Huh7 cells were transfected with LipoD293 (SignaGen Laboratories) with 3C substrate, 3C protease (derived from CVV3) and a Renilla transfection control plasmid (siCheck). Protease and target constructs were generated using protocols previously described (Dial et al., Viruses 11, (2019), incorporated by reference). The cells were combined with firefly substrate (Bright-Glo; Promega) followed by subsequent Renilla (Stop and Glo; Promega) luciferase substrate 24 hours post transfection. Assays were performed using the manufacturer's recommendations (Promega) and a Veritas Microplate Luminometer (Turner BioSystems) was used to quantify the results.

Influenza a Infection

MDCK-SIAT1-TMPRSS2 cells were infected with Influenza A/Puerto Rico/8/1934 (PR8) at an MOI of 0.01 TCID50/cell. Following a 1 hour adsorption, virus was removed and the cells were washed. Viral growth medium was added with either masitinib or DMSO to a final concentration of 10 μM. Supernatants were harvested and clarified at 20 hours post infection. The supernatants were titrated using TCID on MDCK-SIAT1-TMPRSS2 cells.

LCMV Infection

One day prior to infection, A549 cells were seeded in 12 well dishes (80,000 cells per well). Cells were infected with rLCMV at an MOI of 0.01 for one hour at 37° C. The inoculum was removed and cells were overlaid with 1 mL of complete media containing masitinib or DMSO only control. Supernatants were harvested at 48 hours after infection, were clarified and titrated by a previously described immuno-focus assay (Graham et al., medRxiv 2020.07.15.20154443 (2020). doi:10.1101/2020.07.15.20154443 and Ziegler et al., Gen. Virol. 97: 2084 2089 (2016), each incorporated by reference herein), using a mouse anti-LCMV nucleoprotein antibody (1-1.3) and a peroxidase-labeled goat antimouse antibody (SeraCare).

Measles Virus Inhibition Assay

Vero cells were infected with a luciferase-expressing measles virus at an MOI of 0.01 for 90 minutes. The inoculum was removed and added fresh medium containing masitinib or DMSO to the cells for further culture. Three days later, firefly luciferase activity was measured by adding 0.5 mM of D-Luciferin to each well and was quantified with an Infinite M200 Pro multimode microplate reader.

Statistical Analyses

For all experiment described, the size of the sample (n) refers to independent biological samples tested. All analyses were performed in Matlab. Multiple-comparison corrections was performed using the FDR method.

Results

A drug repurposing screen against the human beta coronavirus OC43 identifies masitinib that is effective against SARS-CoV-2.

A library of 1,900 clinically used drugs was screened, the drugs either approved for human use or having extensive safety data in humans (Phase 2 or 3 clinical trials), for their ability to inhibit OC43 infection of the human lung epithelial cell line A549 (expressing an H2BmRuby nuclear reporter). One day after plating, cells were infected at an MOI of 0.3, incubated at 33° C. for 1 hour and drugs were added to a final concentration of 10 PM. Cells were then incubated at 33° C. for 4 days, fixed and stained for the presence of the viral nucleoprotein. The cells were imaged at day 0 (following drug addition) and day 4 (after staining) to determine the drugs effect on cell growth and OC43 infection.

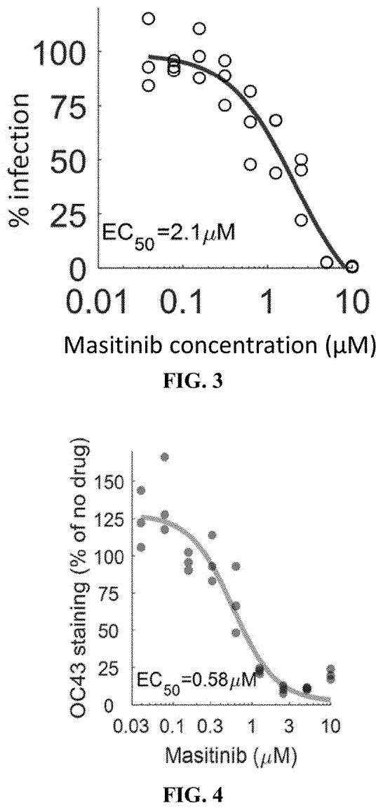

The screen was repeated twice and identified 108 drugs, including masitinib, that significantly reduced OC43 infection without significant cellular toxicity. Overall agreement between the two repeats was high (R2=0.81). Of the top hits, 29 drugs were reselected for further validation, among which masitinib. Masitinib thus inhibited OC43 infection in a dose-dependent manner, with an EC50 value (drug concentration required to reduce infection by 50%) against OC43 infection of 2.1 μM (FIG. 3).

Furthermore, FIG. 4 presents results showing that masitinib inhibits OC43 replication in primary human airway epithelial cells with an EC50 of 0.58 μM.

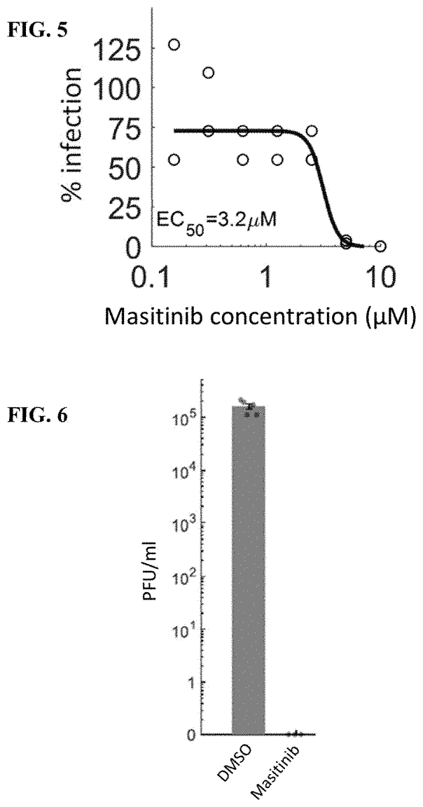

The EC50 value against SARS-CoV-2 infection was determined for masitinib. In a high biocontainment (BSL3) facility, A549 cells over-expressing the angiotensin-converting enzyme 2 (ACE2) receptor were treated with masitinib for 2 hours, infected with SARS-CoV-2 at an MOI of 0.5, incubated for 2 days, fixed, and stained for the viral spike protein (as a marker of SARS-CoV-2 infection). After staining, the cells were imaged under a microscope to quantify the fraction of infected cells. Masitinib inhibited SARS-CoV-2 infection in a dose-dependent manner with an EC50 value of 3.2 μM (FIG. 5).

The effect of masitinib on the production of viable progeny viruses was evaluated. Cells were treated with 10 μM of masitinib for 2 hours, infected at an MOI of 0.5 and the supernatant collected 2 days after for titration (FIG. 6). Masitinib completely eliminated SARS-CoV-2 progeny production (>5-logs decrease).

The screen thus identified masitinib as a safe-in-human drug that is able to inhibit both OC43 and SARS-CoV-2 infection in vitro.

Masitinib is a Bone-Fide 3CLpro Inhibitor

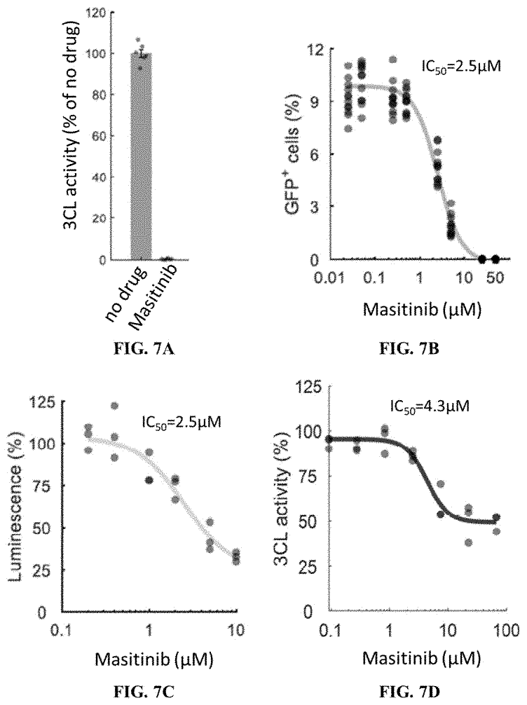

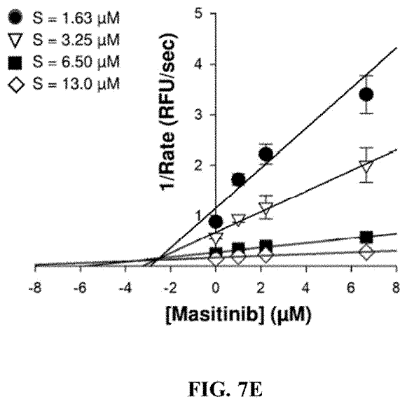

The ability of masitinib to inhibit the SARS-CoV-2 main protease (also known as 3CLpro, M^{pro} and nsp5) was investigated. 3CLpro is indispensable for the viral replication cycle and is well conserved among coronaviruses. The ability of masitinib was tested with regard to inhibition of 3CLpro activity in 293T cells transfected with a FlipGFP reporter system (Anand et al., Science, 300: 1763-1767 (2003), incorporated by reference herein) at a single concentration of 10 μM. In this assay, 3CLpro cleavage of the FlipGFP reporter is needed to produce GFP fluorescence, and thus the level of GFP+ cells reports on 3CLpro activity. As shown on FIG. 7A, masitinib showed a statistically significant decrease in the percentage of GFP-expressing cells. Masitinib completely inhibited 3CLpro activity.

Therefore, the IC50 value (the drug concentration that causes a 50% reduction in enzymatic activity) of masitinib inhibition was determined for 3CLpro activity in two distinct cellular assays: the same FlipGFP reporter assay described above (FIG. 7B), as well as a luciferase reporter assay adapted for SARS-CoV-224 (FIG. 7C). These assays determined the IC50 value to be 2.5 μM (FIG. 7B-C), similar to the EC50 values determined against OC43 infection (2.1 μM, FIG. 3) and SARS-CoV-2 infection (3.2 μM, FIG. 5), suggesting that masitinib inhibition of coronavirus infection is achieved by inhibiting 3CLpro activity.

The IC50 value of masitinib was evaluated using a cell-free assay with purified 3CLpro and a fluorescent substrate, in which fluorescent signal is uncaged upon 3CLpro proteolytic activity (FIG. 7D, IC50=4.3 μM. See above for complete description of the assay). This assay strongly suggested that direct interaction of masitinib with the viral protease is responsible for its effects on SARS-CoV-2 replication.

Finally, in vitro characterization of masitinib inhibition of 3CL was measured in the presence of different substrate concentrations. Results are in FIG. 7E. Masitinib is a competitive inhibitor of 3CL activity with a Ki value of 2.58 μM.

Masitinib Inhibits 3CLpro by Directly Binding to its Active Site

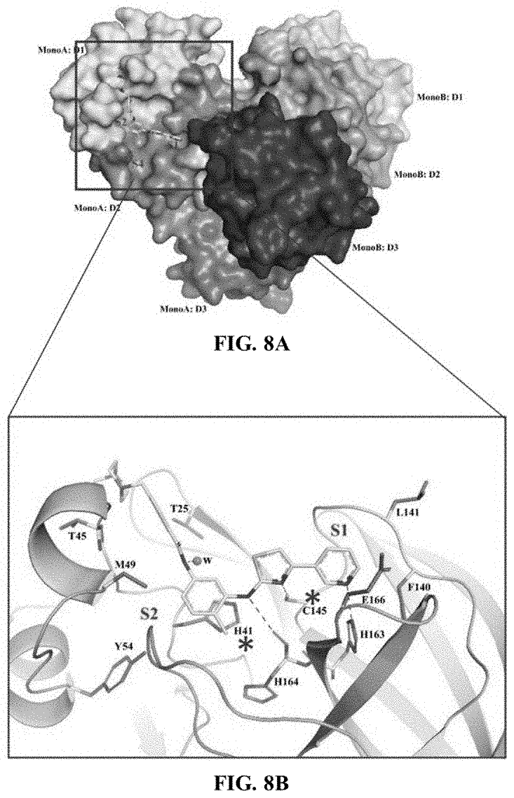

To obtain further mechanistic understating of the mode of 3CLpro inhibition by masitinib, the high-resolution structure of masitinib-bound 3CLpro was determined using X-ray crystallography (FIGS. 8A-B). The structure indicates that masitinib binds non-covalently into the shallow, elongated grove between domains I and II of 3CLpro and is crossing the 3CLpro active site. The enzyme is a dimer and both active sites are occupied by masitinib. Specifically, the pyridine ring of masitinib packs into the S1 substrate pocket of 3CLpro peptide recognition site. In addition to hydrophobic and Van der Waals interactions between the ring and its surrounding pocket-forming residues, it forms a hydrogen bond with His163, located at the bottom of the S1 pocket. The aminothiazole ring of masitinib forms a hydrogen bond with the carbonyl of His164 and interacts directly with the key catalytic residue—Cys145. The second catalytic residue, His41, forms a nearly perfect π-π stacking with the hydrophobic toluene ring of masitinib that occupies the S2 binding pocket. These three active groups (pyridine, aminothiazole and toluene rings) contribute the majority of interactions between masitinib and 3CLpro, bind the key active site residues and effectively block the peptide substrate access to the protease catalytic dyad, thus preventing polyprotein cleavage.

Taken together, the results show that masitinib, originally designed as a tyrosine-kinase inhibitor and considered for treatment of a number of human diseases, harbors potent anti-coronavirus activity through its direct binding to and inhibition of the virus main protease.

Masitinib Blocks the Replication of Picornaviruses Through the Inhibition of their 3C Protease

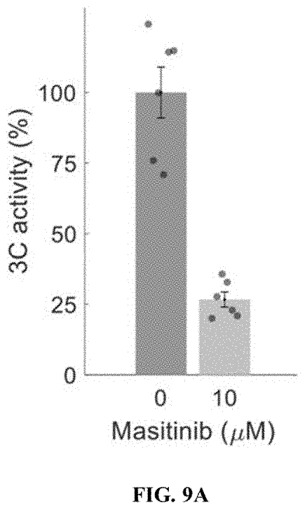

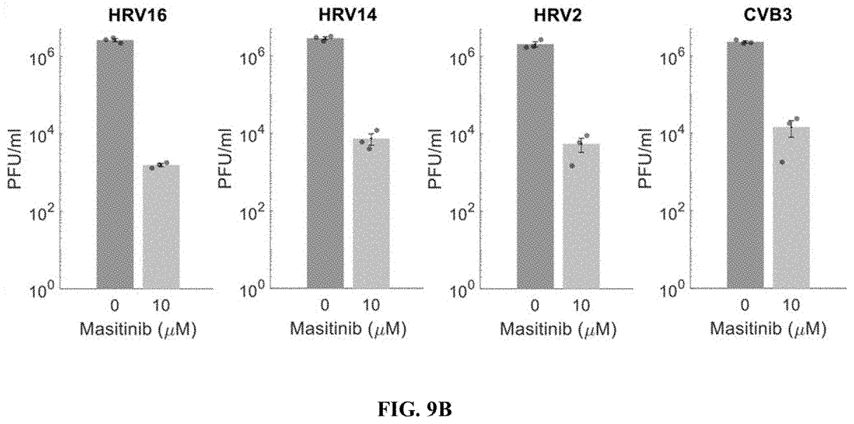

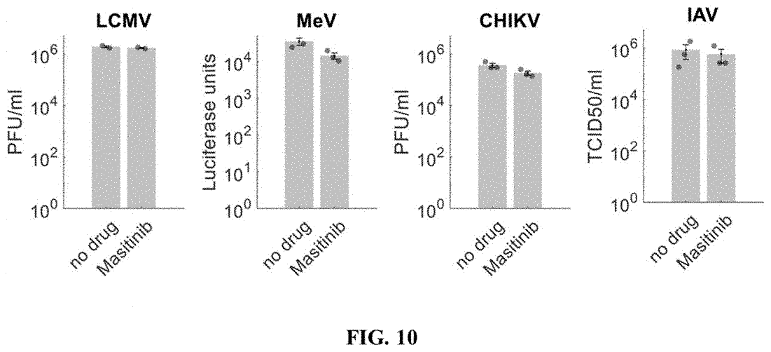

Since masitinib directly binds and inhibits the coronaviruses 3CL protease, an investigation into its effectiveness against the 3C protease of picornaviruses (human pathogens that cause a range of diseases including meningitis, hepatitis, and poliomyelitis) was conducted given the extensive structural homology and substrate specificity shared between these viral proteases. Using a luciferase reporter assay (Dial et al., Viruses, 11: (2019), incorporated by reference herein), it was found that masitinib significantly inhibited the activity of the 3C protease in cells (FIG. 9A). Masitinib was also effective in blocking the replication of multiple picornaviruses, i.e., coxsackievirus B3 (CVB3) and human rhinoviruses 2, 14 and 16 (HRV2, HRV14, HRV16) (FIG. 9B) but not of other RNA viruses, i.e., influenza A virus (IAV, Orthomyxoviridae), measles virus (MeV, Paramyxoviridae), lymphocytic choriomeningitis virus (LCMV) and Chikungunya virus (CHIKV, Togaviridae) (FIG. 10). Thus, masitinib is able to inhibit multiple corona- and picorna-viruses, but not other RNA viruses that do not rely on a 3CL-like protease to complete their life cycle.

While masitinib binds to 3CLpro in a non-covalent manner, it shows better efficacy against SARS-CoV-2 replication in vitro than covalent, pre-clinical, 3CLpro inhibitor 13b (Zhang et al., Science, 368: 409-412 (2020)). Furthermore, while the EC50 value of masitinib is higher than that of two other, pre-clinical, covalent inhibitors, 11a and 11b (Dai et al., Science, 368: 1331-1335 (2020)), it showed superior inhibition of progeny production at 10 μM (over 5-logs for masitinib, compared to 2-logs for 11a and 11b).

### Example 5: In Vivo Inhibitory Effect of Masitinib on SARS-CoV-2

Materials and Methods

Seven week old female Tg (K18-hACE2) 2Prlmn (Jackson Laboratories, Bar Harbor, Me.) mice were challenged with 2×10⁴ pfu in 50 μL of USA-WA1/2020 SARS-CoV-2 (2019-nCoV) by intranasal delivery. Mock-infected female mice received 50 μL of PBS in lieu of viral challenge. Mice were treated twice daily (i.e., bid), starting 12 hours after inoculation, with either PBS or masitinib ranging in concentration from 25 mg/kg to 50 mg/kg in a volume of 100 μL via intraperitoneal injection. Mice were followed twice daily for clinical symptoms and weight loss for 6 days post-challenge. Categories included in clinical scoring included posture and appearance of fur (piloerection) (0-3, with the lower score indicating the better posture and appearance), and development of respiratory distress (0-3, with the lower score indicating the lesser respiratory distress). At day four and day six post-challenge, five mice from each treatment group were sacrificed and the lungs and nasal turbinates collected to evaluate viral load. All mouse work was approved by the institutional animal care and use committee, and all procedures were performed in a certified animal biosafety level three laboratory.

Results

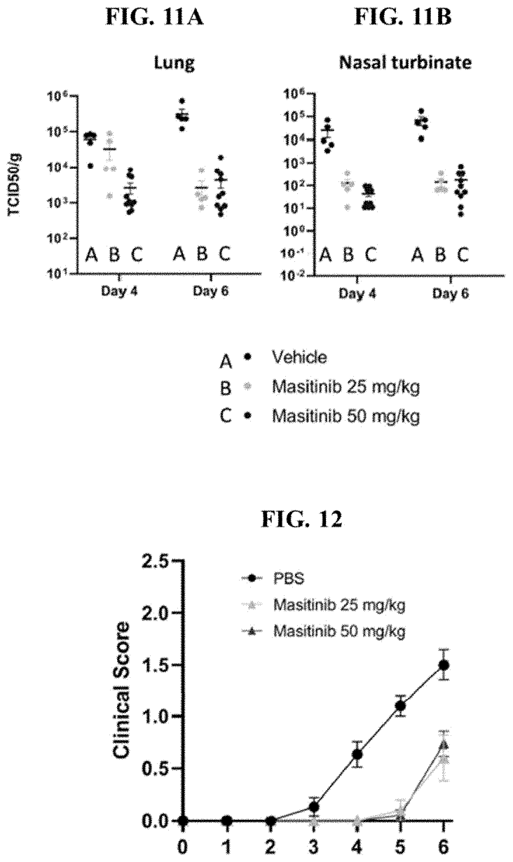

SARS-CoV-2 viral loads in mice were measured, 4 and 6 days post infection with SARS-CoV-2. Mice were treated with masitinib (25 or 50 mg/kg, bid, ip) or PBS. As shown in FIGS. 11A-B, masitinib induced a significant decrease in SARS-CoV-2 viral load, both in the lung and the nasal turbinates.

Clinical score of mice was measured, 1-6 days post infection with SARS-CoV-2. Mice were treated with masitinib (25 or 50 mg/kg, bid, ip) or PBS. As shown in FIG. 12, masitinib (either at a dose of 25 mg/kg, bid, ip or at a dose 50 mg/kg, bid, ip) induced a significant decrease in the clinical score, that is to say a significant betterment of the mice clinical status.

The results presented herein thus demonstrate an effective in vivo therapeutic effect of masitinib in the treatment of a SARS-CoV-2 infection.

1-15. (canceled)

16. A method for treating a severe acute respiratory syndrome coronavirus 2 (SARS-CoV-2) infection causing coronavirus disease 2019 (COVID-19) in a subject in need thereof, comprising administering to the subject masitinib, or a pharmaceutically acceptable salt or solvate thereof.

17. The method according to claim 16, wherein said masitinib, or the pharmaceutically acceptable salt or solvate thereof, is administered in combination with isoquercetin.

18. The method according to claim 18, wherein said masitinib, or the pharmaceutically acceptable salt or solvate thereof, is administered in combination with a dose of isoquercetin ranging from about 0.4 g/day to about 2 g/day.

19. The method according to claim 16, wherein the pharmaceutically acceptable salt of masitinib is masitinib mesilate.

20. The method according to claim 16, wherein said masitinib, or the pharmaceutically acceptable salt or solvate thereof, is administered at a dose ranging from about 1 mg/kg/day to about 12 mg/kg/day (mg per kilo body weight per day).

21. The method according to claim 16, wherein said masitinib, or the pharmaceutically acceptable salt or solvate thereof, is administered at an initial dose of about 3 mg/kg/day during at least 2 days, and at a dose of about 4.5 mg/kg/day thereafter, with each dose escalation being subjected to toxicity controls.

22. The method according to claim 16, wherein the subject presents at least one risk factor that may lead to an increased risk of developing COVID-19.

23. The method according to claim 16, wherein the subject is suffering from mild-to-moderate COVID-19.

24. The method according to claim 16, wherein the subject is suffering from moderate COVID-19.

25. The method according to claim 16, wherein the subject is suffering from severe COVID-19.

26. The method according to claim 16, wherein the subject is suffering from critical COVID-19.

27. The method according to claim 16, wherein the subject is suffering from COVID-19 and has a score on the World Health Organization (WHO) 10-point progression scale of COVID-19 ranging from 2 to 9.

28. The method according to claim 27, wherein the subject is suffering from COVID-19 and has a score on the WHO 10-point progression scale of COVID-19 of 2 or 3.

29. The method according to claim 16, wherein the subject is suffering from COVID-19 and has a score on the WHO 10-point progression scale of COVID-19 ranging from 4 to 6.

30. The method according to claim 29, wherein the subject is suffering from COVID-19 and has a score on the WHO 10-point progression scale of COVID-19 of 4 or 5.

31. The method according to claim 16, wherein the subject is suffering from COVID-19 and has a score on the modified WHO 7-point progression scale of COVID-19 ranging from 2 to 6.

32. The method according to claim 31, wherein the subject is suffering from COVID-19 and has a score on the modified WHO 7-point progression scale of COVID-19 ranging from 2 to 5.

33. The method according to claim 31, wherein the subject is suffering from COVID-19 and has a score on the modified WHO 7-point progression scale of COVID-19 of 4 or 5.

34. The method according to claim 16, wherein said masitinib, or the pharmaceutically acceptable salt or solvate thereof, is administered with at least one further pharmaceutically active agent.

35. The method according to claim 34, wherein the at least one further pharmaceutically active agent is selected from the group consisting of antiviral agents, anti-interleukin 6 (anti-IL6) agents, protease inhibitors, Janus-associated kinase (JAK) inhibitors, BXT-25, brilacidin, dehydroandrographolide succinate, APNO1, fingolimod, methylprednisolone, thalidomide, bevacizumab, sildenafil citrate, interferon, carrimycin, and any mixes thereof.
